(19) United States
(12) Patent Application Publication
(10) Pub. No.: US 2026/0250775 A1
(43) Pub. Date: Aug. 27, 2026

(54) ASSAY FOR THE DETECTION OF OROPHARYNGEAL CANCER

(71) Applicant: H. LEE MOFFITT CANCER CENTER AND RESEARCH INSTITUTE, INC., Tampa, FL (US)

(72) Inventor: Anna R. GIULIANO, Tampa, FL (US)

(21) Appl. No.: 19/161,493

(22) PCT Filed: Mar. 1, 2024
(86) PCT No.: PCT/US2024/018001

§ 371 Date: Sep. 2, 2025

Related U.S. Application Data
(60) Provisional application No. 63/449,665, filed on Mar. 3, 2023.
(60) Provisional application No. 63/452,352, filed on Mar. 15, 2023.
(60) Provisional application No. 63/455,829, filed on Mar. 30, 2023.

(51) Int. Cl. C12Q 1/6886; C12Q 1/70

(52) U.S. Cl. CPC C12Q 1/6886; C12Q 1/708; C12Q 2600/154; G01N 2800/26

(57) ABSTRACT

Disclosed herein is an assay comprising a probe panel to detect the methylation status of 12 genes and one CPG site. Also disclosed herein are methods for using an oral wash and the probe panel to detect the presence of oropharyngeal cancer based on the methylation status of the genes and the CPG site.

### CROSS REFERENCE TO RELATED APPLICATIONS

This application claims the benefit of U.S. Provisional Application No. 63/449,665, filed on Mar. 3, 2023, U.S. Provisional Application No. 63/452,352, filed on Mar. 15, 2023, and U.S. Provisional Application No. 63/455,829, filed on Mar. 30, 2023, application which are incorporated herein by reference in their entireties.

### STATEMENT OF GOVERNMENT SUPPORT

This invention was made with government support under Grant No. R21DE024816 awarded by National Institutes of Health. The government has certain rights in the invention.

### REFERENCE TO SEQUENCE LISTING

The sequence listing submitted on Mar. 1, 2024, as an .XML file entitled “10110-440WO1_ST26.XML” created on Mar. 1, 2024, and having a file size of 18,980 bytes is hereby incorporated by reference pursuant to 37 C.F.R. § 1.52(e)(5).

### I. BACKGROUND

As oropharyngeal cancer (OPC) associated with human papillomavirus (HPV) is increasing for middle-aged men in developed countries, the need for early diagnosis is also increasing. While survival is better for HPV-associated OPCs, metastasis is still likely and 13-25% of patients experience recurrence within two years and is often incurable regardless of HPV status. Due to a lack of observable pre-cancerous lesions, primary prevention through screening is not possible for OPC. Thus, the detection of early OPC (T1-T2, N0-N1; small tumors with only a single ipsilateral positive node <3 cm) is key to reducing treatment severity and adverse outcomes after diagnosis. What are needed are new assays for the detection of OPC.

### II. SUMMARY

Disclosed are assays and biomarker panels for the detection of oropharyngeal cancer and methods of using the same for the detection and treatment of oropharyngeal cancer. Such assays and biomarker panels are an effective tool to detect OPC early and prevent complications of treatment associated with later diagnosis.

In one aspect, disclosed herein are assays for the detection of oropharyngeal cancer in a subject comprising probes to detect methylation at CpG sites in one or more genes in a tissue sample; wherein the genes comprise PAPD5, ST8SIA5, SNORD115-20, NCRNA00164; MIR663B, DDX42, RASA3, HMGB2, ARMC6, EPB41L3 (such as, for example, CpG probes cg25191818, cg19367172, cg12176286, cg19487745, cg20791412, cg11125104, cg27131607, cg14500050, cg14705778, cg04025917, cg06459104, and cg00619915); and wherein a change in the amount of methylation of 3 or more CpG sites relative to a normal control indicates the presence of oropharyngeal cancer (for example, an increase in methylation at CpG sites of one or more of PAPD5, ST8SIA5, SNORD115-20, NCRNA00164; MIR663B, DDX42, RASA3, and/or ARMC6; and/or a decrease at HMGB2 or EPB41L3). In one aspect, the assay further comprises a probe for the detection of EPB41L3 CpG site 438 and/or an HPV SPF₁₀ PCR-DEIA-LiPA₂₅ line probe for the detection of human papilloma virus 16 infection or human papilloma virus 18 infection.

Also disclosed herein are methods of diagnosing and/or detecting the presence of an oropharyngeal cancer in a subject comprising obtaining a tissue sample from the subject and applying the tissue sample to the assay of any preceding aspect. For example, disclosed herein are methods of diagnosing and/or detecting the presence of an oropharyngeal cancer in a subject comprising i) obtaining a tissue sample from the subject (including, but not limited performing an oral wash, gargle, throat swab, scrape, or biopsy on the subject, or collecting saliva from the subject) and ii) measuring the methylation at CpG sites of one or more genes comprising PAPD5, ST8SIA5, SNORD115-20, NCRNA00164; MIR663B, DDX42, RASA3, HMGB2, ARMC6, and/or EPB41L3; wherein a change in the amount of methylation of 3 or more CpG sites relative to a normal control indicates the presence of oropharyngeal cancer (for example, an increase in methylation at CpG sites of one or more of PAPD5, ST8SIA5, SNORD115-20, NCRNA00164; MIR663B, DDX42, RASA3, and/or ARMC6; and/or a decrease at HMGB2 or EPB41L3). In one aspect, the methods can further comprise the detection of methylation at EPB41L3 CpG site 438 and/or an HPV16 and/or HPV18 infection.

In one aspect, disclosed herein are methods of diagnosing and/or detecting the presence of an oropharyngeal cancer of any preceding aspect, wherein the detection of CpG sites is accomplished using the CpG probes cg25191818, cg19367172, cg12176286, cg19487745, cg20791412, cg11125104, cg27131607, cg14500050, cg14705778, cg04025917, cg06459104, and cg00619915. For example, the methylation change can be an increase in methylation detected with one or more of the probes cg25191818, cg19367172, cg12176286, cg19487745, cg11125104, cg27131607, cg14500050, cg04025917, and/or cg0061991; and/or a decrease detected in methylation of one or more of the probes cg20791412, cg14705778, and/or cg06459104.

Also disclosed herein are methods of diagnosing and/or detecting the presence of an oropharyngeal cancer of any preceding aspect, wherein HPV16 and/or HPV16 infection is detected using an HPV SPF₁₀ PCR-DEIA-LiPA₂₅ line probe.

In one aspect, disclosed herein are methods of treating, inhibiting, decreasing, reducing, ameliorating and/or preventing oropharyngeal cancer or a metastasis thereof in a subject comprising i) obtaining a tissue sample from the subject, ii) applying the tissue sample to the assay of any preceding aspect and/or performing the methods of diagnosing and/or detecting the presence of an oropharyngeal cancer of any preceding aspect, and iii) treating the oropharyngeal cancer when detected. For example, disclosed herein are methods of treating, inhibiting, decreasing, reducing, ameliorating and/or preventing oropharyngeal cancer or a metastasis thereof in a subject comprising i) obtaining a tissue sample from the subject (including, but not limited performing an oral wash, gargle, throat swab, scrape, or biopsy on the subject, or collecting saliva from the subject); ii) measuring the methylation at CpG sites of one or more genes comprising PAPD5, ST8SIA5, SNORD115-20, NCRNA00164; MIR663B, DDX42, RASA3, HMGB2, ARMC6, and/or EPB41L3; wherein a change in the amount of methylation of 3 or more CpG sites relative to a normal control indicates the presence of oropharyngeal cancer (for example, an increase in methylation at CpG sites of one or more of PAPD5, ST8SIA5, SNORD115-20, NCRNA00164; MIR663B, DDX42, RASA3, and/or ARMC6; and/or a decrease at HMGB2 or EPB41L3), and iii) treating the oropharyngeal cancer when detected. In one aspect, the methods can further comprise the detection of methylation at EPB41L3 CpG site 438 and/or an HPV16 and/or HPV18 infection.

Also disclosed herein are methods of treating, inhibiting, decreasing, reducing, ameliorating and/or preventing oropharyngeal cancer or a metastasis thereof of any preceding aspect, wherein the detection of CpG sites is accomplished using the CpG probes cg25191818, cg19367172, cg12176286, cg19487745, cg20791412, cg11125104, cg27131607, cg14500050, cg14705778, cg04025917, cg06459104, and cg00619915. For example, the methylation change can be an increase in methylation detected with one or more of the probes cg25191818, cg19367172, cg12176286, cg19487745, cg11125104, cg27131607, cg14500050, cg04025917, and/or cg0061991; and/or a decrease detected in methylation of one or more of the probes cg20791412, cg14705778, and/or cg06459104.

In one aspect, disclosed herein are methods of treating, inhibiting, decreasing, reducing, ameliorating and/or preventing oropharyngeal cancer or a metastasis thereof of any preceding aspect, wherein HPV16 and/or HPV16 infection is detected using an HPV SPF₁₀ PCR-DEIA-LiPA₂₅ line probe.

Also disclosed herein are methods of treating, inhibiting, decreasing, reducing, ameliorating and/or preventing oropharyngeal cancer or a metastasis thereof of any preceding aspect, wherein the cancer is treated with the administration of an anti-cancer agent and/or radiation.

### III. BRIEF DESCRIPTION OF THE DRAWINGS

The accompanying drawings, which are incorporated in and constitute a part of this specification, illustrate several embodiments and together with the description illustrate the disclosed compositions and methods.

FIG. 1 shows the distribution of the Area Under the Curve (AUC) values. Four different variable selection methodologies were performed to determine which one resulted best prediction ability (i.e. higher AUCs on the test set). Each experiment consisted of 50 rounds with a random selection of a training set and a validation set, 66.67% and 33.33% respectively. The variables selected by the given method were used to build a LASSO model, which was subsequently used to predict the probability of a sample belonging to an early case or control. Experiments consisted of: (1) Variables selected only from discovery using Illumina EPIC array probes; (2) Step 1 and manual addition of the results from pyrosequencing three EPB41L3 CpG sites; (3) Step 1, 2, and manual addition HPV16 status; (4); Steps 1-3 and addition of the Illumina identified EPB41L3 CpG site.

FIG. 2A shows ROC Curve for the Training Model generated from the 14 selected markers. A single bootstrap sample was used for training the model, resulting in an AUC of 0.978.

FIG. 2B shows ROC Curve for the Test Model generated from a corresponding set of “left-out” samples using boostratp re-sampling, resulting in a validation AUC of 0.935.

FIG. 3 shows the number of non-zero coefficients per variable. In the variable selection process for the final model, variables could have been given a non-zero coefficient anywhere from one to fifty times. The variables were ranked according to how many times each one received a non-zero coefficient in descending order. Using the heuristic that a variable should be given a non-zero coefficient at least half of the time, we started with a soft cut-off of 25, the solid red line. Since there was something of a plateau around 25, the dotted red lines, the variable that was given a non-zero coefficient 23 times was also included in the final model for a total of 14 variables.

FIG. 4 shows a detailed schema of the processes used to build the biomarker panel. Actions on the left represent processes that were part of cross-validation, while actions on the right represent individual steps towards identification of biomarkers to include in the panel. Steps 1-5 correspond with those described in detail in the methods.

### IV. DETAILED DESCRIPTION

Before the present compounds, compositions, articles, devices, and/or methods are disclosed and described, it is to be understood that they are not limited to specific synthetic methods or specific recombinant biotechnology methods unless otherwise specified, or to particular reagents unless otherwise specified, as such may, of course, vary. It is also to be understood that the terminology used herein is for the purpose of describing particular embodiments only and is not intended to be limiting.

### A. DEFINITIONS

As used in the specification and the appended claims, the singular forms “a,” “an” and “the” include plural referents unless the context clearly dictates otherwise. Thus, for example, reference to “a pharmaceutical carrier” includes mixtures of two or more such carriers, and the like.

Ranges can be expressed herein as from “about” one particular value, and/or to “about” another particular value. When such a range is expressed, another embodiment includes from the one particular value and/or to the other particular value. Similarly, when values are expressed as approximations, by use of the antecedent “about,” it will be understood that the particular value forms another embodiment. It will be further understood that the endpoints of each of the ranges are significant both in relation to the other endpoint, and independently of the other endpoint. It is also understood that there are a number of values disclosed herein, and that each value is also herein disclosed as “about” that particular value in addition to the value itself. For example, if the value “10” is disclosed, then “about 10” is also disclosed. It is also understood that when a value is disclosed that “less than or equal to” the value, “greater than or equal to the value” and possible ranges between values are also disclosed, as appropriately understood by the skilled artisan. For example, if the value “10” is disclosed the “less than or equal to 10” as well as “greater than or equal to 10” is also disclosed. It is also understood that the throughout the application, data is provided in a number of different formats, and that this data, represents endpoints and starting points, and ranges for any combination of the data points. For example, if a particular data point “10” and a particular data point 15 are disclosed, it is understood that greater than, greater than or equal to, less than, less than or equal to, and equal to 10 and 15 are considered disclosed as well as between 10 and 15. It is also understood that each unit between two particular units are also disclosed. For example, if 10 and 15 are disclosed, then 11, 12, 13, and 14 are also disclosed.

In this specification and in the claims which follow, reference will be made to a number of terms which shall be defined to have the following meanings:

“Optional” or “optionally” means that the subsequently described event or circumstance may or may not occur, and that the description includes instances where said event or circumstance occurs and instances where it does not.

An “increase” can refer to any change that results in a greater amount of a symptom, disease, composition, condition or activity. An increase can be any individual, median, or average increase in a condition, symptom, activity, composition in a statistically significant amount. Thus, the increase can be a 1, 2, 3, 4, 5, 6, 7, 8, 9, 10, 15, 20, 25, 30, 35, 40, 45, 50, 55, 60, 65, 70, 75, 80, 85, 90, 95, or 100% increase so long as the increase is statistically significant.

A “decrease” can refer to any change that results in a smaller amount of a symptom, disease, composition, condition, or activity. A substance is also understood to decrease the genetic output of a gene when the genetic output of the gene product with the substance is less relative to the output of the gene product without the substance. Also for example, a decrease can be a change in the symptoms of a disorder such that the symptoms are less than previously observed. A decrease can be any individual, median, or average decrease in a condition, symptom, activity, composition in a statistically significant amount. Thus, the decrease can be a 1, 2, 3, 4, 5, 6, 7, 8, 9, 10, 15, 20, 25, 30, 35, 40, 45, 50, 55, 60, 65, 70, 75, 80, 85, 90, 95, or 100% decrease so long as the decrease is statistically significant.

“Inhibit,” “inhibiting,” and “inhibition” mean to decrease an activity, response, condition, disease, or other biological parameter. This can include but is not limited to the complete ablation of the activity, response, condition, or disease. This may also include, for example, a 10% reduction in the activity, response, condition, or disease as compared to the native or control level. Thus, the reduction can be a 10, 20, 30, 40, 50, 60, 70, 80, 90, 100%, or any amount of reduction in between as compared to native or control levels.

By “reduce” or other forms of the word, such as “reducing” or “reduction,” is meant lowering of an event or characteristic (e.g., tumor growth). It is understood that this is typically in relation to some standard or expected value, in other words it is relative, but that it is not always necessary for the standard or relative value to be referred to. For example, “reduces tumor growth” means reducing the rate of growth of a tumor relative to a standard or a control.

By “prevent” or other forms of the word, such as “preventing” or “prevention,” is meant to stop a particular event or characteristic, to stabilize or delay the development or progression of a particular event or characteristic, or to minimize the chances that a particular event or characteristic will occur. Prevent does not require comparison to a control as it is typically more absolute than, for example, reduce. As used herein, something could be reduced but not prevented, but something that is reduced could also be prevented. Likewise, something could be prevented but not reduced, but something that is prevented could also be reduced. It is understood that where reduce or prevent are used, unless specifically indicated otherwise, the use of the other word is also expressly disclosed.

The term “subject” refers to any individual who is the target of administration or treatment. The subject can be a vertebrate, for example, a mammal. In one aspect, the subject can be human, non-human primate, bovine, equine, porcine, canine, or feline. The subject can also be a guinea pig, rat, hamster, rabbit, mouse, or mole. Thus, the subject can be a human or veterinary patient. The term “patient” refers to a subject under the treatment of a clinician, e.g., physician.

The term “therapeutically effective” refers to the amount of the composition used is of sufficient quantity to ameliorate one or more causes or symptoms of a disease or disorder. Such amelioration only requires a reduction or alteration, not necessarily elimination.

The term “treatment” refers to the medical management of a patient with the intent to cure, ameliorate, stabilize, or prevent a disease, pathological condition, or disorder. This term includes active treatment, that is, treatment directed specifically toward the improvement of a disease, pathological condition, or disorder, and also includes causal treatment, that is, treatment directed toward removal of the cause of the associated disease, pathological condition, or disorder. In addition, this term includes palliative treatment, that is, treatment designed for the relief of symptoms rather than the curing of the disease, pathological condition, or disorder; preventative treatment, that is, treatment directed to minimizing or partially or completely inhibiting the development of the associated disease, pathological condition, or disorder; and supportive treatment, that is, treatment employed to supplement another specific therapy directed toward the improvement of the associated disease, pathological condition, or disorder.

“Biocompatible” generally refers to a material and any metabolites or degradation products thereof that are generally non-toxic to the recipient and do not cause significant adverse effects to the subject.

“Comprising” is intended to mean that the compositions, methods, etc. include the recited elements, but do not exclude others. “Consisting essentially of” when used to define compositions and methods, shall mean including the recited elements, but excluding other elements of any essential significance to the combination. Thus, a composition consisting essentially of the elements as defined herein would not exclude trace contaminants from the isolation and purification method and pharmaceutically acceptable carriers, such as phosphate buffered saline, preservatives, and the like. “Consisting of” shall mean excluding more than trace elements of other ingredients and substantial method steps for administering the compositions provided and/or claimed in this disclosure. Embodiments defined by each of these transition terms are within the scope of this disclosure.

A “control” is an alternative subject or sample used in an experiment for comparison purposes. A control can be “positive” or “negative.”

“Effective amount” of an agent refers to a sufficient amount of an agent to provide a desired effect. The amount of agent that is “effective” will vary from subject to subject, depending on many factors such as the age and general condition of the subject, the particular agent or agents, and the like. Thus, it is not always possible to specify a quantified “effective amount.” However, an appropriate “effective amount” in any subject case may be determined by one of ordinary skill in the art using routine experimentation. Also, as used herein, and unless specifically stated otherwise, an “effective amount” of an agent can also refer to an amount covering both therapeutically effective amounts and prophylactically effective amounts. An “effective amount” of an agent necessary to achieve a therapeutic effect may vary according to factors such as the age, sex, and weight of the subject. Dosage regimens can be adjusted to provide the optimum therapeutic response. For example, several divided doses may be administered daily or the dose may be proportionally reduced as indicated by the exigencies of the therapeutic situation.

A “pharmaceutically acceptable” component can refer to a component that is not biologically or otherwise undesirable, i.e., the component may be incorporated into a pharmaceutical formulation provided by the disclosure and administered to a subject as described herein without causing significant undesirable biological effects or interacting in a deleterious manner with any of the other components of the formulation in which it is contained. When used in reference to administration to a human, the term generally implies the component has met the required standards of toxicological and manufacturing testing or that it is included on the Inactive Ingredient Guide prepared by the U.S. Food and Drug Administration.

“Pharmaceutically acceptable carrier” (sometimes referred to as a “carrier”) means a carrier or excipient that is useful in preparing a pharmaceutical or therapeutic composition that is generally safe and non-toxic and includes a carrier that is acceptable for veterinary and/or human pharmaceutical or therapeutic use. The terms “carrier” or “pharmaceutically acceptable carrier” can include, but are not limited to, phosphate buffered saline solution, water, emulsions (such as an oil/water or water/oil emulsion) and/or various types of wetting agents. As used herein, the term “carrier” encompasses, but is not limited to, any excipient, diluent, filler, salt, buffer, stabilizer, solubilizer, lipid, stabilizer, or other material well known in the art for use in pharmaceutical formulations and as described further herein.

“Pharmacologically active” (or simply “active”), as in a “pharmacologically active” derivative or analog, can refer to a derivative or analog (e.g., a salt, ester, amide, conjugate, metabolite, isomer, fragment, etc.) having the same type of pharmacological activity as the parent compound and approximately equivalent in degree.

“Therapeutic agent” refers to any composition that has a beneficial biological effect. Beneficial biological effects include both therapeutic effects, e.g., treatment of a disorder or other undesirable physiological condition, and prophylactic effects, e.g., prevention of a disorder or other undesirable physiological condition (e.g., a non-immunogenic cancer). The terms also encompass pharmaceutically acceptable, pharmacologically active derivatives of beneficial agents specifically mentioned herein, including, but not limited to, salts, esters, amides, proagents, active metabolites, isomers, fragments, analogs, and the like. When the terms “therapeutic agent” is used, then, or when a particular agent is specifically identified, it is to be understood that the term includes the agent per se as well as pharmaceutically acceptable, pharmacologically active salts, esters, amides, proagents, conjugates, active metabolites, isomers, fragments, analogs, etc.

“Therapeutically effective amount” or “therapeutically effective dose” of a composition (e.g. a composition comprising an agent) refers to an amount that is effective to achieve a desired therapeutic result. In some embodiments, a desired therapeutic result is the control of type I diabetes. In some embodiments, a desired therapeutic result is the control of obesity. Therapeutically effective amounts of a given therapeutic agent will typically vary with respect to factors such as the type and severity of the disorder or disease being treated and the age, gender, and weight of the subject. The term can also refer to an amount of a therapeutic agent, or a rate of delivery of a therapeutic agent (e.g., amount over time), effective to facilitate a desired therapeutic effect, such as pain relief. The precise desired therapeutic effect will vary according to the condition to be treated, the tolerance of the subject, the agent and/or agent formulation to be administered (e.g., the potency of the therapeutic agent, the concentration of agent in the formulation, and the like), and a variety of other factors that are appreciated by those of ordinary skill in the art. In some instances, a desired biological or medical response is achieved following administration of multiple dosages of the composition to the subject over a period of days, weeks, or years.

“Primers” are a subset of probes which are capable of supporting some type of enzymatic manipulation and which can hybridize with a target nucleic acid such that the enzymatic manipulation can occur. A primer can be made from any combination of nucleotides or nucleotide derivatives or analogs available in the art which do not interfere with the enzymatic manipulation.

“Probes” are molecules capable of interacting with a target nucleic acid, typically in a sequence specific manner, for example through hybridization. The hybridization of nucleic acids is well understood in the art and discussed herein. Typically a probe can be made from any combination of nucleotides or nucleotide derivatives or analogs available in the art.

Throughout this application, various publications are referenced. The disclosures of these publications in their entireties are hereby incorporated by reference into this application in order to more fully describe the state of the art to which this pertains. The references disclosed are also individually and specifically incorporated by reference herein for the material contained in them that is discussed in the sentence in which the reference is relied upon.

### B. COMPOSITIONS

Disclosed are the components to be used to prepare the disclosed compositions as well as the compositions themselves to be used within the methods disclosed herein. These and other materials are disclosed herein, and it is understood that when combinations, subsets, interactions, groups, etc. of these materials are disclosed that while specific reference of each various individual and collective combinations and permutation of these compounds may not be explicitly disclosed, each is specifically contemplated and described herein. For example, if a particular oropharyngeal detection assay is disclosed and discussed and a number of modifications that can be made to a number of molecules including the oropharyngeal detection assay are discussed, specifically contemplated is each and every combination and permutation of oropharyngeal detection assay and the modifications that are possible unless specifically indicated to the contrary. Thus, if a class of molecules A, B, and C are disclosed as well as a class of molecules D, E, and F and an example of a combination molecule, A-D is disclosed, then even if each is not individually recited each is individually and collectively contemplated meaning combinations, A-E, A-F, B-D, B-E, B-F, C-D, C-E, and C-F are considered disclosed. Likewise, any subset or combination of these is also disclosed. Thus, for example, the sub-group of A-E, B-F, and C-E would be considered disclosed. This concept applies to all aspects of this application including, but not limited to, steps in methods of making and using the disclosed compositions. Thus, if there are a variety of additional steps that can be performed it is understood that each of these additional steps can be performed with any specific embodiment or combination of embodiments of the disclosed methods.

As oropharyngeal cancer (OPC) associated with human papillomavirus (HPV) is increasing for middle-aged men in developed countries, the need for early diagnosis is also increasing. While survival is better for HPV-associated OPCs, metastasis is still likely and 13-25% of patients experience recurrence within two years and is often incurable regardless of HPV status. Due to a lack of observable pre-cancerous lesions, primary prevention through screening is not possible for OPC. Thus, the detection of early OPC (T1-T2, N0-N1; small tumors with only a single ipsilateral positive node <3 cm) is key to reducing treatment severity and adverse outcomes after diagnosis.

Methylation of the host tumor suppressor gene, EPB41L3, and the HPV16 genome assessed from oral gargles in pretreatment, male OPC cases (n=228) was significantly (p<0.0001) higher among cases compared to controls and predicted cases with an AUC of 0.82. However, the observed prediction in early OPC cases alone (T1-T2, N0-N1; small tumors with a single ipsilateral node <3 cm) was lower (AUC=0.78), indicating the need for additional biomarkers to improve screening performance.

Methylation across the genome at 850,000 CpG sites can now be assessed by a standardized array. Therefore, to expand on the site-specific methylation and to explore additional biomarkers that can differentiate early cases from late cases and controls, we performed a genome-wide methylation among the same study population to identify additional markers of early HPV-associated OPC and created a methylation assay to more accurately detect oropharyngeal cancer.

In one aspect, disclosed herein are assays for the detection of oropharyngeal cancer in a subject comprising probes to detect methylation at CpG sites in one or more genes in a tissue sample; wherein the genes comprise PAPD5, ST8SIA5, SNORD115-20, NCRNA00164; MIR663B, DDX42, RASA3, HMGB2, ARMC6, EPB41L3 (such as, for example, CpG probes cg25191818, cg19367172, cg12176286, cg19487745, cg20791412, cg11125104, cg27131607, cg14500050, cg14705778, cg04025917, cg06459104, and cg00619915); and wherein a change in the amount of methylation of 3 or more CpG sites relative to a normal control indicates the presence of oropharyngeal cancer (for example, an increase in methylation at CpG sites of one or more of PAPD5, ST8SIA5, SNORD115-20, NCRNA00164; MIR663B, DDX42, RASA3, and/or ARMC6; and/or a decrease at HMGB2 or EPB41L3). For example, the methylation change can be an increase in methylation detected with one or more of the probes cg25191818, cg19367172, cg12176286, cg19487745, cg11125104, cg27131607, cg14500050, cg04025917, and/or cg0061991; and/or a decrease detected in methylation of one or more of the probes cg20791412, cg14705778, and/or cg06459104. In one aspect, the assay further comprises a probe for the detection of EPB41L3 CpG site 438 and/or an HPV SPF₁₀ PCR-DEIA-LiPA₂₅ line probe for the detection of human papilloma virus 16 infection or human papilloma virus 18 infection.

### C. METHOD OF TREATING CANCER

It is understood and herein contemplated that the disclosed methods can be used in the detection of oropharyngeal cancer. Thus, disclosed herein are methods of diagnosing and/or detecting the presence of an oropharyngeal cancer in a subject comprising obtaining a tissue sample from the subject and applying the tissue sample to the assay disclosed herein. For example, disclosed herein are methods of diagnosing and/or detecting the presence of an oropharyngeal cancer in a subject comprising i) obtaining a tissue sample from the subject (including, but not limited performing an oral wash, gargle, throat swab, scrape, or biopsy on the subject, or collecting saliva from the subject) and ii) measuring the methylation at CpG sites of one or more genes comprising PAPD5, ST8SIA5, SNORD115-20, NCRNA00164; MIR663B, DDX42, RASA3, HMGB2, ARMC6, and/or EPB41L3; wherein a change in the amount of methylation of 3 or more CpG sites relative to a normal control indicates the presence of oropharyngeal cancer (for example, an increase in methylation at CpG sites of one or more of PAPD5, ST8SIA5, SNORD115-20, NCRNA00164; MIR663B, DDX42, RASA3, and/or ARMC6; and/or a decrease at HMGB2 or EPB41L3). In one aspect, the methods can further comprise the detection of methylation at EPB41L3 CpG site 438 and/or an HPV16 and/or HPV18 infection.

Also disclosed herein are methods of diagnosing and/or detecting the presence of an oropharyngeal cancer wherein the detection of CpG sites is accomplished using the CpG probes cg25191818, cg19367172, cg12176286, cg19487745, cg20791412, cg11125104, cg27131607, cg14500050, cg14705778, cg04025917, cg06459104, and cg00619915. For example, the methylation change can be an increase in methylation detected with one or more of the probes cg25191818, cg19367172, cg12176286, cg19487745, cg11125104, cg27131607, cg14500050, cg04025917, and/or cg0061991; and/or a decrease detected in methylation of one or more of the probes cg20791412, cg14705778, and/or cg06459104.

In one aspect, disclosed herein are methods of diagnosing and/or detecting the presence of an oropharyngeal cancer of any preceding aspect, wherein HPV16 and/or HPV16 infection is detected using an HPV SPF₁₀ PCR-DEIA-LiPA₂₅ line probe.

The disclosed methods can be used to treat any disease where uncontrolled cellular proliferation occurs such as cancers such as oropharyngeal cancers including squamous cell carcinomas of the mouth, throat, larynx; esophageal carcinoma; and head and neck carcinoma.

In one aspect, disclosed herein are methods of treating, inhibiting, decreasing, reducing, ameliorating and/or preventing oropharyngeal cancer or a metastasis thereof in a subject comprising i) obtaining a tissue sample from the subject, ii) applying the tissue sample to the assay disclosed herein and/or performing the methods of diagnosing and/or detecting the presence of an oropharyngeal cancer disclosed herein, and iii) treating the oropharyngeal cancer when detected. For example, disclosed herein are methods of treating, inhibiting, decreasing, reducing, ameliorating and/or preventing oropharyngeal cancer or a metastasis thereof in a subject comprising i) obtaining a tissue sample from the subject (including, but not limited performing an oral wash, gargle, throat swab, scrape, or biopsy on the subject, or collecting saliva from the subject); ii) measuring the methylation at CpG sites of one or more genes comprising PAPD5, ST8SIA5, SNORD115-20, NCRNA00164; MIR663B, DDX42, RASA3, HMGB2, ARMC6, and/or EPB41L3; wherein a change in the amount of methylation of 3 or more CpG sites relative to a normal control indicates the presence of oropharyngeal cancer (for example, an increase in methylation at CpG sites of one or more of PAPD5, ST8SIA5, SNORD115-20, NCRNA00164; MIR663B, DDX42, RASA3, and/or ARMC6; and/or a decrease at HMGB2 or EPB41L3), and iii) treating the oropharyngeal cancer when detected. In one aspect, the methods can further comprise the detection of methylation at EPB41L3 CpG site 438 and/or an HPV16 and/or HPV18 infection.

Also disclosed herein are methods of treating, inhibiting, decreasing, reducing, ameliorating and/or preventing oropharyngeal cancer or a metastasis thereof, wherein the detection of CpG sites is accomplished using the CpG probes cg25191818, cg19367172, cg12176286, cg19487745, cg20791412, cg11125104, cg27131607, cg14500050, cg14705778, cg04025917, cg06459104, and cg00619915. For example, the methylation change can be an increase in methylation detected with one or more of the probes cg25191818, cg19367172, cg12176286, cg19487745, cg11125104, cg27131607, cg14500050, cg04025917, and/or cg0061991; and/or a decrease detected in methylation of one or more of the probes cg20791412, cg14705778, and/or cg06459104.

In one aspect, disclosed herein are methods of treating, inhibiting, decreasing, reducing, ameliorating and/or preventing oropharyngeal cancer or a metastasis thereof, wherein HPV16 and/or HPV16 infection is detected using an HPV SPF₁₀ PCR-DEIA-LiPA₂₅ line probe.

Also disclosed herein are methods of treating, inhibiting, decreasing, reducing, ameliorating and/or preventing oropharyngeal cancer or a metastasis thereof, wherein the cancer is treated with the administration of an anti-cancer agent and/or radiation. It is understood and herein contemplated that the disclosed treatment regimens can used alone or in combination with any anti-cancer therapy known in the art including, but not limited to Abemaciclib, Abiraterone Acetate, Abitrexate (Methotrexate), Abraxane (Paclitaxel Albumin-stabilized Nanoparticle Formulation), ABVD, ABVE, ABVE-PC, AC, AC-T, Adcetris (Brentuximab Vedotin), ADE, Ado-Trastuzumab Emtansine, Adriamycin (Doxorubicin Hydrochloride), Afatinib Dimaleate, Afinitor (Everolimus), Akynzeo (Netupitant and Palonosetron Hydrochloride), Aldara (Imiquimod), Aldesleukin, Alecensa (Alectinib), Alectinib, Alemtuzumab, Alimta (Pemetrexed Disodium), Aliqopa (Copanlisib Hydrochloride), Alkeran for Injection (Melphalan Hydrochloride), Alkeran Tablets (Melphalan), Aloxi (Palonosetron Hydrochloride), Alunbrig (Brigatinib), Ambochlorin (Chlorambucil), Amboclorin Chlorambucil), Amifostine, Aminolevulinic Acid, Anastrozole, Aprepitant, Aredia (Pamidronate Disodium), Arimidex (Anastrozole), Aromasin (Exemestane), Arranon (Nelarabine), Arsenic Trioxide, Arzerra (Ofatumumab), Asparaginase Erwinia chrysanthemi, Atezolizumab, Avastin (Bevacizumab), Avelumab, Axitinib, Azacitidine, Bavencio (Avelumab), BEACOPP, Becenum (Carmustine), Beleodaq (Belinostat), Belinostat, Bendamustine Hydrochloride, BEP, Besponsa (Inotuzumab Ozogamicin), Bevacizumab, Bexarotene, Bexxar (Tositumomab and Iodine I 131 Tositumomab), Bicalutamide, BiCNU (Carmustine), Bleomycin, Blinatumomab, Blincyto (Blinatumomab), Bortezomib, Bosulif (Bosutinib), Bosutinib, Brentuximab Vedotin, Brigatinib, BuMel, Busulfan, Busulfex (Busulfan), Cabazitaxel, Cabometyx (Cabozantinib-S-Malate), Cabozantinib-S-Malate, CAF, Campath (Alemtuzumab), Camptosar, (Irinotecan Hydrochloride), Capecitabine, CAPOX, Carac (Fluorouracil—Topical), Carboplatin, CARBOPLATIN-TAXOL, Carfilzomib, Carmubris (Carmustine), Carmustine, Carmustine Implant, Casodex (Bicalutamide), CEM, Ceritinib, Cerubidine (Daunorubicin Hydrochloride), Cervarix (Recombinant HPV Bivalent Vaccine), Cetuximab, CEV, Chlorambucil, CHLORAMBUCIL-PREDNISONE, CHOP, Cisplatin, Cladribine, Clafen (Cyclophosphamide), Clofarabine, Clofarex (Clofarabine), Clolar (Clofarabine), CMF, Cobimetinib, Cometriq (Cabozantinib-S-Malate), Copanlisib Hydrochloride, COPDAC, COPP, COPP-ABV, Cosmegen (Dactinomycin), Cotellic (Cobimetinib), Crizotinib, CVP, Cyclophosphamide, Cyfos (Ifosfamide), Cyramza (Ramucirumab), Cytarabine, Cytarabine Liposome, Cytosar-U (Cytarabine), Cytoxan (Cyclophosphamide), Dabrafenib, Dacarbazine, Dacogen (Decitabine), Dactinomycin, Daratumumab, Darzalex (Daratumumab), Dasatinib, Daunorubicin Hydrochloride, Daunorubicin Hydrochloride and Cytarabine Liposome, Decitabine, Defibrotide Sodium, Defitelio (Defibrotide Sodium), Degarelix, Denileukin Diftitox, Denosumab, DepoCyt (Cytarabine Liposome), Dexamethasone, Dexrazoxane Hydrochloride, Dinutuximab, Docetaxel, Doxil (Doxorubicin Hydrochloride Liposome), Doxorubicin Hydrochloride, Doxorubicin Hydrochloride Liposome, Dox-SL (Doxorubicin Hydrochloride Liposome), DTIC-Dome (Dacarbazine), Durvalumab, Efudex (Fluorouracil—Topical), Elitek (Rasburicase), Ellence (Epirubicin Hydrochloride), Elotuzumab, Eloxatin (Oxaliplatin), Eltrombopag Olamine, Emend (Aprepitant), Empliciti (Elotuzumab), Enasidenib Mesylate, Enzalutamide, Epirubicin Hydrochloride, EPOCH, Erbitux (Cetuximab), Eribulin Mesylate, Erivedge (Vismodegib), Erlotinib Hydrochloride, Erwinaze (Asparaginase Erwinia chrysanthemi), Ethyol (Amifostine), Etopophos (Etoposide Phosphate), Etoposide, Etoposide Phosphate, Evacet (Doxorubicin Hydrochloride Liposome), Everolimus, Evista, (Raloxifene Hydrochloride), Evomela (Melphalan Hydrochloride), Exemestane, 5-FU (Fluorouracil Injection), 5-FU (Fluorouracil—Topical), Fareston (Toremifene), Farydak (Panobinostat), Faslodex (Fulvestrant), FEC, Femara (Letrozole), Filgrastim, Fludara (Fludarabine Phosphate), Fludarabine Phosphate, Fluoroplex (Fluorouracil—Topical), Fluorouracil Injection, Fluorouracil—Topical, Flutamide, Folex (Methotrexate), Folex PFS (Methotrexate), FOLFIRI, FOLFIRI-BEVACIZUMAB, FOLFIRI-CETUXIMAB, FOLFIRINOX, FOLFOX, Folotyn (Pralatrexate), FU-LV, Fulvestrant, Gardasil (Recombinant HPV Quadrivalent Vaccine), Gardasil 9 (Recombinant HPV Nonavalent Vaccine), Gazyva (Obinutuzumab), Gefitinib, Gemcitabine Hydrochloride, GEMCITABINE-CISPLATIN, GEMCITABINE-OXALIPLATIN, Gemtuzumab Ozogamicin, Gemzar (Gemcitabine Hydrochloride), Gilotrif (Afatinib Dimaleate), Gleevec (Imatinib Mesylate), Gliadel (Carmustine Implant), Gliadel wafer (Carmustine Implant), Glucarpidase, Goserelin Acetate, Halaven (Eribulin Mesylate), Hemangeol (Propranolol Hydrochloride), Herceptin (Trastuzumab), HPV Bivalent Vaccine, Recombinant, HPV Nonavalent Vaccine, Recombinant, HPV Quadrivalent Vaccine, Recombinant, Hycamtin (Topotecan Hydrochloride), Hydrea (Hydroxyurea), Hydroxyurea, Hyper-CVAD, Ibrance (Palbociclib), Ibritumomab Tiuxetan, Ibrutinib, ICE, Iclusig (Ponatinib Hydrochloride), Idamycin (Idarubicin Hydrochloride), Idarubicin Hydrochloride, Idelalisib, Idhifa (Enasidenib Mesylate), Ifex (Ifosfamide), Ifosfamide, Ifosfamidum (Ifosfamide), IL-2 (Aldesleukin), Imatinib Mesylate, Imbruvica (Ibrutinib), Imfinzi (Durvalumab), Imiquimod, Imlygic (Talimogene Laherparepvec), Inlyta (Axitinib), Inotuzumab Ozogamicin, Interferon Alfa-2b, Recombinant, Interleukin-2 (Aldesleukin), Intron A (Recombinant Interferon Alfa-2b), Iodine 1131 Tositumomab and Tositumomab, Ipilimumab, Iressa (Gefitinib), Irinotecan Hydrochloride, Irinotecan Hydrochloride Liposome, Istodax (Romidepsin), Ixabepilone, Ixazomib Citrate, Ixempra (Ixabepilone), Jakafi (Ruxolitinib Phosphate), JEB, Jevtana (Cabazitaxel), Kadcyla (Ado-Trastuzumab Emtansine), Keoxifene (Raloxifene Hydrochloride), Kepivance (Palifermin), Keytruda (Pembrolizumab), Kisqali (Ribociclib), Kymriah (Tisagenlecleucel), Kyprolis (Carfilzomib), Lanreotide Acetate, Lapatinib Ditosylate, Lartruvo (Olaratumab), Lenalidomide, Lenvatinib Mesylate, Lenvima (Lenvatinib Mesylate), Letrozole, Leucovorin Calcium, Leukeran (Chlorambucil), Leuprolide Acetate, Leustatin (Cladribine), Levulan (Aminolevulinic Acid), Linfolizin (Chlorambucil), LipoDox (Doxorubicin Hydrochloride Liposome), Lomustine, Lonsurf (Trifluridine and Tipiracil Hydrochloride), Lupron (Leuprolide Acetate), Lupron Depot (Leuprolide Acetate), Lupron Depot-Ped (Leuprolide Acetate), Lynparza (Olaparib), Marqibo (Vincristine Sulfate Liposome), Matulane (Procarbazine Hydrochloride), Mechlorethamine Hydrochloride, Megestrol Acetate, Mekinist (Trametinib), Melphalan, Melphalan Hydrochloride, Mercaptopurine, Mesna, Mesnex (Mesna), Methazolastone (Temozolomide), Methotrexate, Methotrexate LPF (Methotrexate), Methylnaltrexone Bromide, Mexate (Methotrexate), Mexate-AQ (Methotrexate), Midostaurin, Mitomycin C, Mitoxantrone Hydrochloride, Mitozytrex (Mitomycin C), MOPP, Mozobil (Plerixafor), Mustargen (Mechlorethamine Hydrochloride), Mutamycin (Mitomycin C), Myleran (Busulfan), Mylosar (Azacitidine), Mylotarg (Gemtuzumab Ozogamicin), Nanoparticle Paclitaxel (Paclitaxel Albumin-stabilized Nanoparticle Formulation), Navelbine (Vinorelbine Tartrate), Necitumumab, Nelarabine, Neosar (Cyclophosphamide), Neratinib Maleate, Nerlynx (Neratinib Maleate), Netupitant and Palonosetron Hydrochloride, Neulasta (Pegfilgrastim), Neupogen (Filgrastim), Nexavar (Sorafenib Tosylate), Nilandron (Nilutamide), Nilotinib, Nilutamide, Ninlaro (Ixazomib Citrate), Niraparib Tosylate Monohydrate, Nivolumab, Nolvadex (Tamoxifen Citrate), Nplate (Romiplostim), Obinutuzumab, Odomzo (Sonidegib), OEPA, Ofatumumab, OFF, Olaparib, Olaratumab, Omacetaxine Mepesuccinate, Oncaspar (Pegaspargase), Ondansetron Hydrochloride, Onivyde (Irinotecan Hydrochloride Liposome), Ontak (Denileukin Diftitox), Opdivo (Nivolumab), OPPA, Osimertinib, Oxaliplatin, Paclitaxel, Paclitaxel Albumin-stabilized Nanoparticle Formulation, PAD, Palbociclib, Palifermin, Palonosetron Hydrochloride, Palonosetron Hydrochloride and Netupitant, Pamidronate Disodium, Panitumumab, Panobinostat, Paraplat (Carboplatin), Paraplatin (Carboplatin), Pazopanib Hydrochloride, PCV, PEB, Pegaspargase, Pegfilgrastim, Peginterferon Alfa-2b, PEG-Intron (Peginterferon Alfa-2b), Pembrolizumab, Pemetrexed Disodium, Perjeta (Pertuzumab), Pertuzumab, Platinol (Cisplatin), Platinol-AQ (Cisplatin), Plerixafor, Pomalidomide, Pomalyst (Pomalidomide), Ponatinib Hydrochloride, Portrazza (Necitumumab), Pralatrexate, Prednisone, Procarbazine Hydrochloride, Proleukin (Aldesleukin), Prolia (Denosumab), Promacta (Eltrombopag Olamine), Propranolol Hydrochloride, Provenge (Sipuleucel-T), Purinethol (Mercaptopurine), Purixan (Mercaptopurine), Radium 223 Dichloride, Raloxifene Hydrochloride, Ramucirumab, Rasburicase, R-CHOP, R-CVP, Recombinant Human Papillomavirus (HPV) Bivalent Vaccine, Recombinant Human Papillomavirus (HPV) Nonavalent Vaccine, Recombinant Human Papillomavirus (HPV) Quadrivalent Vaccine, Recombinant Interferon Alfa-2b, Regorafenib, Relistor (Methylnaltrexone Bromide), R-EPOCH, Revlimid (Lenalidomide), Rheumatrex (Methotrexate), Ribociclib, R-ICE, Rituxan (Rituximab), Rituxan Hycela (Rituximab and Hyaluronidase Human), Rituximab, Rituximab and, Hyaluronidase Human, Rolapitant Hydrochloride, Romidepsin, Romiplostim, Rubidomycin (Daunorubicin Hydrochloride), Rubraca (Rucaparib Camsylate), Rucaparib Camsylate, Ruxolitinib Phosphate, Rydapt (Midostaurin), Sclerosol Intrapleural Aerosol (Talc), Siltuximab, Sipuleucel-T, Somatuline Depot (Lanreotide Acetate), Sonidegib, Sorafenib Tosylate, Sprycel (Dasatinib), STANFORD V, Sterile Talc Powder (Talc), Steritalc (Talc), Stivarga (Regorafenib), Sunitinib Malate, Sutent (Sunitinib Malate), Sylatron (Peginterferon Alfa-2b), Sylvant (Siltuximab), Synribo (Omacetaxine Mepesuccinate), Tabloid (Thioguanine), TAC, Tafinlar (Dabrafenib), Tagrisso (Osimertinib), Talc, Talimogene Laherparepvec, Tamoxifen Citrate, Tarabine PFS (Cytarabine), Tarceva (Erlotinib Hydrochloride), Targretin (Bexarotene), Tasigna (Nilotinib), Taxol (Paclitaxel), Taxotere (Docetaxel), Tecentriq, (Atezolizumab), Temodar (Temozolomide), Temozolomide, Temsirolimus, Thalidomide, Thalomid (Thalidomide), Thioguanine, Thiotepa, Tisagenlecleucel, Tolak (Fluorouracil—Topical), Topotecan Hydrochloride, Toremifene, Torisel (Temsirolimus), Tositumomab and Iodine 1131 Tositumomab, Totect (Dexrazoxane Hydrochloride), TPF, Trabectedin, Trametinib, Trastuzumab, Treanda (Bendamustine Hydrochloride), Trifluridine and Tipiracil Hydrochloride, Trisenox (Arsenic Trioxide), Tykerb (Lapatinib Ditosylate), Unituxin (Dinutuximab), Uridine Triacetate, VAC, Vandetanib, VAMP, Varubi (Rolapitant Hydrochloride), Vectibix (Panitumumab), VeIP, Velban (Vinblastine Sulfate), Velcade (Bortezomib), Velsar (Vinblastine Sulfate), Vemurafenib, Venclexta (Venetoclax), Venetoclax, Verzenio (Abemaciclib), Viadur (Leuprolide Acetate), Vidaza (Azacitidine), Vinblastine Sulfate, Vincasar PFS (Vincristine Sulfate), Vincristine Sulfate, Vincristine Sulfate Liposome, Vinorelbine Tartrate, VIP, Vismodegib, Vistogard (Uridine Triacetate), Voraxaze (Glucarpidase), Vorinostat, Votrient (Pazopanib Hydrochloride), Vyxeos (Daunorubicin Hydrochloride and Cytarabine Liposome), Wellcovorin (Leucovorin Calcium), Xalkori (Crizotinib), Xeloda (Capecitabine), XELIRI, XELOX, Xgeva (Denosumab), Xofigo (Radium 223 Dichloride), Xtandi (Enzalutamide), Yervoy (Ipilimumab), Yondelis (Trabectedin), Zaltrap (Ziv-Aflibercept), Zarxio (Filgrastim), Zejula (Niraparib Tosylate Monohydrate), Zelboraf (Vemurafenib), Zevalin (Ibritumomab Tiuxetan), Zinecard (Dexrazoxane Hydrochloride), Ziv-Aflibercept, Zofran (Ondansetron Hydrochloride), Zoladex (Goserelin Acetate), Zoledronic Acid, Zolinza (Vorinostat), Zometa (Zoledronic Acid), Zydelig (ldelalisib), Zykadia (Ceritinib), and/or Zytiga (Abiraterone Acetate). The treatment methods can include or further include checkpoint inhibitors including, but are not limited to antibodies that block PD-1 (such as, for example, Nivolumab (BMS-936558 or MDX1106), pemibralizunab, CT-011, MK-3475), PD-L1 (such as, for example, atezolizumab, avelumab, durvalunab, MDX-1105 (BMS-936559), MPDL3280A, or MSB0010718C), PD-L2 (such as, for example, rHIgM12B7), CTLA-4 (such as, for example, Ipilimumab (MDX-010), Tremelimumab (CP-675,206)), IDO, B7-H3 (such as, for example, MGA271, MGD009, omburtamab), B7-H4, B7-H3, T cell immunoreceptor with Ig and ITIM domains (TIGIT) (such as, for example BMS-986207, OMP-313M32, MK-7684, AB-154, ASP-8374, MTIG7192A, or PVSRIPO), CD96, B- and T-lymphocyte attenuator (BTLA), V-domain Ig suppressor of T cell activation (VISTA)(such as, for example, JNJ-61610588, CA-170), TIM3 (such as, for example, TSR-022, MBG453, Sym023, INCAGN2390, LY3321367, BMS-986258, SHR-1702, R07121661), LAG-3 (such as, for example, BMS-986016, LAG525, MK-4280, REGN3767, TSR-033, BI754111, Sym022, FS118, MGD013, and Immutep)

### 1. Immunoassays and Fluorochromes

The steps of various useful immunodetection methods have been described in the scientific literature, such as, e.g., Maggio et al., Enzyme-Immunoassay, (1987) and Nakamura, et al., Enzyme Immunoassays: Heterogeneous and Homogeneous Systems, Handbook of Experimental Immunology, Vol. 1: Immunochemistry, 27.1-27.20 (1986), each of which is incorporated herein by reference in its entirety and specifically for its teaching regarding immunodetection methods. Immunoassays, in their most simple and direct sense, are binding assays involving binding between antibodies and antigen. Many types and formats of immunoassays are known and all are suitable for detecting the disclosed biomarkers. Examples of immunoassays are enzyme linked immunosorbent assays (ELISAs), radioimmunoassays (RIA), radioimmune precipitation assays (RIPA), immunobead capture assays, Western blotting, dot blotting, gel-shift assays, Flow cytometry, protein arrays, multiplexed bead arrays, magnetic capture, in vivo imaging, fluorescence resonance energy transfer (FRET), and fluorescence recovery/localization after photobleaching (FRAP/FLAP).

In general, immunoassays involve contacting a sample suspected of containing a molecule of interest (such as the disclosed biomarkers) with an antibody to the molecule of interest or contacting an antibody to a molecule of interest (such as antibodies to the disclosed biomarkers) with a molecule that can be bound by the antibody, as the case may be, under conditions effective to allow the formation of immunocomplexes. Contacting a sample with the antibody to the molecule of interest or with the molecule that can be bound by an antibody to the molecule of interest under conditions effective and for a period of time sufficient to allow the formation of immune complexes (primary immune complexes) is generally a matter of simply bringing into contact the molecule or antibody and the sample and incubating the mixture for a period of time long enough for the antibodies to form immune complexes with, i.e., to bind to, any molecules (e.g., antigens) present to which the antibodies can bind. In many forms of immunoassay, the sample-antibody composition, such as a tissue section, ELISA plate, dot blot or Western blot, can then be washed to remove any non-specifically bound antibody species, allowing only those antibodies specifically bound within the primary immune complexes to be detected.

Immunoassays can include methods for detecting or quantifying the amount of a molecule of interest (such as the disclosed biomarkers or their antibodies) in a sample, which methods generally involve the detection or quantitation of any immune complexes formed during the binding process. In general, the detection of immunocomplex formation is well known in the art and can be achieved through the application of numerous approaches. These methods are generally based upon the detection of a label or marker, such as any radioactive, fluorescent, biological or enzymatic tags or any other known label.

As used herein, a label can include a fluorescent dye, a member of a binding pair, such as biotin/streptavidin, a metal (e.g., gold), or an epitope tag that can specifically interact with a molecule that can be detected, such as by producing a colored substrate or fluorescence. Substances suitable for detectably labeling proteins include fluorescent dyes (also known herein as fluorochromes and fluorophores) and enzymes that react with colorometric substrates (e.g., horseradish peroxidase). The use of fluorescent dyes is generally preferred in the practice of the invention as they can be detected at very low amounts. Furthermore, in the case where multiple antigens are reacted with a single array, each antigen can be labeled with a distinct fluorescent compound for simultaneous detection. Labeled spots on the array are detected using a fluorimeter, the presence of a signal indicating an antigen bound to a specific antibody.

Fluorophores are compounds or molecules that luminesce. Typically fluorophores absorb electromagnetic energy at one wavelength and emit electromagnetic energy at a second wavelength. Representative fluorophores include, but are not limited to, 1,5 IAEDANS; 1,8-ANS; 4-Methylumbelliferone; 5-carboxy-2,7-dichlorofluorescein; 5-Carboxyfluorescein (5-FAM); 5-Carboxynapthofluorescein; 5-Carboxytetramethylrhodamine (5-TAMRA); 5-Hydroxy Tryptamine (5-HAT); 5-ROX (carboxy-X-rhodamine); 6-Carboxyrhodamine 6G; 6-CR 6G; 6-JOE; 7-Amino-4-methylcoumarin; 7-Aminoactinomycin D (7-AAD); 7-Hydroxy-4-I methylcoumarin; 9-Amino-6-chloro-2-methoxyacridine (ACMA); ABQ; Acid Fuchsin; Acridine Orange; Acridine Red; Acridine Yellow; Acriflavin; Acriflavin Feulgen SITSA; Aequorin (Photoprotein); AFPs—AutoFluorescent Protein—(Quantum Biotechnologies) see sgGFP, sgBFP; Alexa Fluor 350™; Alexa Fluor 430™; Alexa Fluor 488™; Alexa Fluor 532™; Alexa Fluor 546™; Alexa Fluor 568™; Alexa Fluor 594™; Alexa Fluor 633™; Alexa Fluor 647™; Alexa Fluor 660™; Alexa Fluor 680™; Alizarin Complexon; Alizarin Red; Allophycocyanin (APC); AMC, AMCA-S; Aminomethylcoumarin (AMCA); AMCA-X; Aminoactinomycin D; Aminocoumarin; Anilin Blue; Anthrocyl stearate; APC-Cy7; APTRA-BTC; APTS; Astrazon Brilliant Red 4G; Astrazon Orange R; Astrazon Red 6B; Astrazon Yellow 7 GLL; Atabrine; ATTO-TAG™ CBQCA; ATTO-TAG™ FQ; Auramine; Aurophosphine G; Aurophosphine; BAO 9 (Bisaminophenyloxadiazole); BCECF (high pH); BCECF (low pH); Berberine Sulphate; Beta Lactamase; BFP blue shifted GFP (Y66H); Blue Fluorescent Protein; BFP/GFP FRET; Bimane; Bisbenzemide; Bisbenzimide (Hoechst); bis-BTC; Blancophor FFG; Blancophor SV; BOBO™-1; BOBO™-3; Bodipy492/515; Bodipy493/503; Bodipy500/510; Bodipy; 505/515; Bodipy 530/550; Bodipy 542/563; Bodipy 558/568; Bodipy 564/570; Bodipy 576/589; Bodipy 581/591; Bodipy 630/650-X; Bodipy 650/665-X; Bodipy 665/676; Bodipy Fl; Bodipy FL ATP; Bodipy Fl-Ceramide; Bodipy R6G SE; Bodipy TMR; Bodipy TMR-X conjugate; Bodipy TMR-X, SE; Bodipy TR; Bodipy TR ATP; Bodipy TR-X SE; BO-PRO™-1; BO-PRO™-3; Brilliant Sulphoflavin FF; BTC; BTC-5N; Calcein; Calcein Blue; Calcium Crimson—; Calcium Green; Calcium Green-1 Ca²⁺ Dye; Calcium Green-2 Ca²⁺; Calcium Green-5N Ca²⁺; Calcium Green-C18 Ca²⁺; Calcium Orange; Calcofluor White; Carboxy-X-rhodamine (5-ROX); Cascade Blue™; Cascade Yellow; Catecholamine; CCF2 (GeneBlazer); CFDA; CFP (Cyan Fluorescent Protein); CFP/YFP FRET; Chlorophyll; Chromomycin A; Chromomycin A; CL-NERF; CMFDA; Coelenterazine; Coelenterazine cp; Coelenterazine f; Coelenterazine fcp; Coelenterazine h; Coelenterazine hcp; Coelenterazine ip; Coelenterazine n; Coelenterazine O; Coumarin Phalloidin; C-phycocyanine; CPM I Methylcoumarin; CTC; CTC Formazan; Cy2™; Cy3.1 8; Cy3.5™; Cy3™; Cy5.1 8; Cy5.5™; Cy5™; Cy7™; Cyan GFP; cyclic AMP Fluorosensor (FiCRhR); Dabcyl; Dansyl; Dansyl Amine; Dansyl Cadaverine; Dansyl Chloride; Dansyl DHPE; Dansyl fluoride; DAPI; Dapoxyl; Dapoxyl 2; Dapoxyl 3′DCFDA; DCFH (Dichlorodihydrofluorescein Diacetate); DDAO; DHR (Dihydorhodamine 123); Di-4-ANEPPS; Di-8-ANEPPS (non-ratio); DiA (4-Di 16-ASP); Dichlorodihydrofluorescein Diacetate (DCFH); DiD-Lipophilic Tracer; DiD (DilC18(5)); DIDS; Dihydorhodamine 123 (DHR); Dil (DilC18(3)); I Dinitrophenol; DiO (DiOC18(3)); DiR; DiR (DilC18(7)); DM-NERF (high pH); DNP; Dopamine; DsRed; DTAF; DY-630-NHS; DY-635-NHS; EBFP; ECFP; EGFP; ELF 97; Eosin; Erythrosin; Erythrosin ITC; Ethidium Bromide; Ethidium homodimer-1 (EthD-1); Euchrysin; EukoLight; Europium (111) chloride; EYFP; Fast Blue; FDA; Feulgen (Pararosaniline); FIF (Formaldehyd Induced Fluorescence); FITC; Flazo Orange; Fluo-3; Fluo-4; Fluorescein (FITC); Fluorescein Diacetate; Fluoro-Emerald; Fluoro-Gold (Hydroxystilbamidine); Fluor-Ruby; FluorX; FM 1-43™; FM 4-46; Fura Red™ (high pH); Fura Red™/Fluo-3; Fura-2; Fura-2/BCECF; Genacryl Brilliant Red B; Genacryl Brilliant Yellow 10GF; Genacryl Pink 3G; Genacryl Yellow 5GF; GeneBlazer; (CCF2); GFP (S65T); GFP red shifted (rsGFP); GFP wild type' non-UV excitation (wtGFP); GFP wild type, UV excitation (wtGFP); GFPuv; Gloxalic Acid; Granular blue; Haematoporphyrin; Hoechst 33258; Hoechst 33342; Hoechst 34580; HPTS; Hydroxycoumarin; Hydroxystilbamidine (FluoroGold); Hydroxytryptamine; Indo-1, high calcium; Indo-1 low calcium; Indodicarbocyanine (DiD); Indotricarbocyanine (DiR); Intrawhite Cf; JC-1; JO JO-1; JO-PRO-1; LaserPro; Laurodan; LDS 751 (DNA); LDS 751 (RNA); Leucophor PAF; Leucophor SF; Leucophor WS; Lissamine Rhodamine; Lissamine Rhodamine B; Calcein/Ethidium homodimer; LOLO-1; LO-PRO-1; Lucifer Yellow; Lyso Tracker Blue; Lyso Tracker Blue-White; Lyso Tracker Green; Lyso Tracker Red; Lyso Tracker Yellow; LysoSensor Blue; LysoSensor Green; LysoSensor Yellow/Blue; Mag Green; Magdala Red (Phloxin B); Mag-Fura Red; Mag-Fura-2; Mag-Fura-5; Mag-lndo-1; Magnesium Green; Magnesium Orange; Malachite Green; Marina Blue; I Maxilon Brilliant Flavin 10 GFF; Maxilon Brilliant Flavin 8 GFF; Merocyanin; Methoxycoumarin; Mitotracker Green FM; Mitotracker Orange; Mitotracker Red; Mitramycin; Monobromobimane; Monobromobimane (mBBr-GSH); Monochlorobimane; MPS (Methyl Green Pyronine Stilbene); NBD; NBD Amine; Nile Red; Nitrobenzoxedidole; Noradrenaline; Nuclear Fast Red; i Nuclear Yellow; Nylosan Brilliant lavin E8G; Oregon Green™; Oregon Green™ 488; Oregon Green™ 500; Oregon Green™ 514; Pacific Blue; Pararosaniline (Feulgen); PBFI; PE-Cy5; PE-Cy7; PerCP; PerCP-Cy5.5; PE-TexasRed (Red 613); Phloxin B (Magdala Red); Phorwite AR; Phorwite BKL; Phorwite Rev; Phorwite RPA; Phosphine 3R; PhotoResist; Phycoerythrin B [PE]; Phycoerythrin R [PE]; PKH26 (Sigma); PKH67; PMIA; Pontochrome Blue Black; POPO-1; POPO-3; PO-PRO-1; PO-I PRO-3; Primuline; Procion Yellow; Propidium lodid (P1); PyMPO; Pyrene; Pyronine; Pyronine B; Pyrozal Brilliant Flavin 7GF; QSY 7; Quinacrine Mustard; Resorufin; RH 414; Rhod-2; Rhodamine; Rhodamine 110; Rhodamine 123; Rhodamine 5 GLD; Rhodamine 6G; Rhodamine B; Rhodamine B 200; Rhodamine B extra; Rhodamine BB; Rhodamine BG; Rhodamine Green; Rhodamine Phallicidine; Rhodamine: Phalloidine; Rhodamine Red; Rhodamine WT; Rose Bengal; R-phycocyanine; R-phycoerythrin (PE); rsGFP; S65A; S65C; S65L; S65T; Sapphire GFP; SBFI; Serotonin; Sevron Brilliant Red 2B; Sevron Brilliant Red 4G; Sevron I Brilliant Red B; Sevron Orange; Sevron Yellow L; sgBFP™ (super glow BFP); sgGFP™ (super glow GFP); SITS (Primuline; Stilbene Isothiosulphonic Acid); SNAFL calcein; SNAFL-1; SNAFL-2; SNARF calcein; SNARFI; Sodium Green; SpectrumAqua; SpectrumGreen; SpectrumOrange; Spectrum Red; SPQ (6-methoxy-N-(3 sulfopropyl) quinolinium); Stilbene; Sulphorhodamine B and C; Sulphorhodamine Extra; SYTO 11; SYTO 12; SYTO 13; SYTO 14; SYTO 15; SYTO 16; SYTO 17; SYTO 18; SYTO 20; SYTO 21; SYTO 22; SYTO 23; SYTO 24; SYTO 25; SYTO 40; SYTO 41; SYTO 42; SYTO 43; SYTO 44; SYTO 45; SYTO 59; SYTO 60; SYTO 61; SYTO 62; SYTO 63; SYTO 64; SYTO 80; SYTO 81; SYTO 82; SYTO 83; SYTO 84; SYTO 85; SYTOX Blue; SYTOX Green; SYTOX Orange; Tetracycline; Tetramethylrhodamine (TRITC); Texas Red™; Texas Red-X™ conjugate; Thiadicarbocyanine (DiSC3); Thiazine Red R; Thiazole Orange; Thioflavin 5; Thioflavin S; Thioflavin TON; Thiolyte; Thiozole Orange; Tinopol CBS (Calcofluor White); TIER; TO-PRO-1; TO-PRO-3; TO-PRO-5; TOTO-1; TOTO-3; TriColor (PE-Cy5); TRITC TetramethylRodaminelsoThioCyanate; True Blue; Tru Red; Ultralite; Uranine B; Uvitex SFC; wt GFP; WW 781; X-Rhodamine; XRITC; Xylene Orange; Y66F; Y66H; Y66W; Yellow GFP; YFP; YO-PRO-1; YO-PRO 3; YOYO-1; YOYO-3; Sybr Green; Thiazole orange (interchelating dyes); semiconductor nanoparticles such as quantum dots; or caged fluorophore (which can be activated with light or other electromagnetic energy source), or a combination thereof.

A modifier unit such as a radionuclide can be incorporated into or attached directly to any of the compounds described herein by halogenation. Examples of radionuclides useful in this embodiment include, but are not limited to, tritium, iodine-125, iodine-131, iodine-123, iodine-124, astatine-210, carbon-11, carbon-14, nitrogen-13, fluorine-18. In another aspect, the radionuclide can be attached to a linking group or bound by a chelating group, which is then attached to the compound directly or by means of a linker. Examples of radionuclides useful in the apset include, but are not limited to, Tc-99m, Re-186, Ga-68, Re-188, Y-90, Sm-153, Bi-212, Cu-67, Cu-64, and Cu-62. Radiolabeling techniques such as these are routinely used in the radiopharmaceutical industry.

The radiolabeled compounds are useful as imaging agents to diagnose neurological disease (e.g., a neurodegenerative disease) or a mental condition or to follow the progression or treatment of such a disease or condition in a mammal (e.g., a human). The radiolabeled compounds described herein can be conveniently used in conjunction with imaging techniques such as positron emission tomography (PET) or single photon emission computerized tomography (SPECT).

Labeling can be either direct or indirect. In direct labeling, the detecting antibody (the antibody for the molecule of interest) or detecting molecule (the molecule that can be bound by an antibody to the molecule of interest) include a label. Detection of the label indicates the presence of the detecting antibody or detecting molecule, which in turn indicates the presence of the molecule of interest or of an antibody to the molecule of interest, respectively. In indirect labeling, an additional molecule or moiety is brought into contact with, or generated at the site of, the immunocomplex. For example, a signal-generating molecule or moiety such as an enzyme can be attached to or associated with the detecting antibody or detecting molecule. The signal-generating molecule can then generate a detectable signal at the site of the immunocomplex. For example, an enzyme, when supplied with suitable substrate, can produce a visible or detectable product at the site of the immunocomplex. ELISAs use this type of indirect labeling.

As another example of indirect labeling, an additional molecule (which can be referred to as a binding agent) that can bind to either the molecule of interest or to the antibody (primary antibody) to the molecule of interest, such as a second antibody to the primary antibody, can be contacted with the immunocomplex. The additional molecule can have a label or signal-generating molecule or moiety. The additional molecule can be an antibody, which can thus be termed a secondary antibody. Binding of a secondary antibody to the primary antibody can form a so-called sandwich with the first (or primary) antibody and the molecule of interest. The immune complexes can be contacted with the labeled, secondary antibody under conditions effective and for a period of time sufficient to allow the formation of secondary immune complexes. The secondary immune complexes can then be generally washed to remove any non-specifically bound labeled secondary antibodies, and the remaining label in the secondary immune complexes can then be detected. The additional molecule can also be or include one of a pair of molecules or moieties that can bind to each other, such as the biotin/avadin pair. In this mode, the detecting antibody or detecting molecule should include the other member of the pair.

Other modes of indirect labeling include the detection of primary immune complexes by a two step approach. For example, a molecule (which can be referred to as a first binding agent), such as an antibody, that has binding affinity for the molecule of interest or corresponding antibody can be used to form secondary immune complexes, as described above. After washing, the secondary immune complexes can be contacted with another molecule (which can be referred to as a second binding agent) that has binding affinity for the first binding agent, again under conditions effective and for a period of time sufficient to allow the formation of immune complexes (thus forming tertiary immune complexes). The second binding agent can be linked to a detectable label or signal-generating molecule or moiety, allowing detection of the tertiary immune complexes thus formed. This system can provide for signal amplification.

Immunoassays that involve the detection of as substance, such as a protein or an antibody to a specific protein, include label-free assays, protein separation methods (i.e., electrophoresis), solid support capture assays, or in vivo detection. Label-free assays are generally diagnostic means of determining the presence or absence of a specific protein, or an antibody to a specific protein, in a sample. Protein separation methods are additionally useful for evaluating physical properties of the protein, such as size or net charge. Capture assays are generally more useful for quantitatively evaluating the concentration of a specific protein, or antibody to a specific protein, in a sample. Finally, in vivo detection is useful for evaluating the spatial expression patterns of the substance, i.e., where the substance can be found in a subject, tissue or cell.

Provided that the concentrations are sufficient, the molecular complexes ([Ab-Ag]n) generated by antibody-antigen interaction are visible to the naked eye, but smaller amounts may also be detected and measured due to their ability to scatter a beam of light. The formation of complexes indicates that both reactants are present, and in immunoprecipitation assays a constant concentration of a reagent antibody is used to measure specific antigen ([Ab-Ag]n), and reagent antigens are used to detect specific antibody ([Ab-Ag]n). If the reagent species is previously coated onto cells (as in hemagglutination assay) or very small particles (as in latex agglutination assay), “clumping” of the coated particles is visible at much lower concentrations. A variety of assays based on these elementary principles are in common use, including Ouchterlony immunodiffusion assay, rocket immunoelectrophoresis, and immunoturbidometric and nephelometric assays. The main limitations of such assays are restricted sensitivity (lower detection limits) in comparison to assays employing labels and, in some cases, the fact that very high concentrations of analyte can actually inhibit complex formation, necessitating safeguards that make the procedures more complex. Some of these Group 1 assays date right back to the discovery of antibodies and none of them have an actual “label” (e.g. Ag-enz). Other kinds of immunoassays that are label free depend on immunosensors, and a variety of instruments that can directly detect antibody-antigen interactions are now commercially available. Most depend on generating an evanescent wave on a sensor surface with immobilized ligand, which allows continuous monitoring of binding to the ligand. Immunosensors allow the easy investigation of kinetic interactions and, with the advent of lower-cost specialized instruments, may in the future find wide application in immunoanalysis.

The use of immunoassays to detect a specific protein can involve the separation of the proteins by electophoresis. Electrophoresis is the migration of charged molecules in solution in response to an electric field. Their rate of migration depends on the strength of the field; on the net charge, size and shape of the molecules and also on the ionic strength, viscosity and temperature of the medium in which the molecules are moving. As an analytical tool, electrophoresis is simple, rapid and highly sensitive. It is used analytically to study the properties of a single charged species, and as a separation technique.

Generally the sample is run in a support matrix such as paper, cellulose acetate, starch gel, agarose or polyacrylamide gel. The matrix inhibits convective mixing caused by heating and provides a record of the electrophoretic run: at the end of the run, the matrix can be stained and used for scanning, autoradiography or storage. In addition, the most commonly used support matrices—agarose and polyacrylamide—provide a means of separating molecules by size, in that they are porous gels. A porous gel may act as a sieve by retarding, or in some cases completely obstructing, the movement of large macromolecules while allowing smaller molecules to migrate freely. Because dilute agarose gels are generally more rigid and easy to handle than polyacrylamide of the same concentration, agarose is used to separate larger macromolecules such as nucleic acids, large proteins and protein complexes. Polyacrylamide, which is easy to handle and to make at higher concentrations, is used to separate most proteins and small oligonucleotides that require a small gel pore size for retardation.

Proteins are amphoteric compounds; their net charge therefore is determined by the pH of the medium in which they are suspended. In a solution with a pH above its isoelectric point, a protein has a net negative charge and migrates towards the anode in an electrical field. Below its isoelectric point, the protein is positively charged and migrates towards the cathode. The net charge carried by a protein is in addition independent of its size—i.e., the charge carried per unit mass (or length, given proteins and nucleic acids are linear macromolecules) of molecule differs from protein to protein. At a given pH therefore, and under non-denaturing conditions, the electrophoretic separation of proteins is determined by both size and charge of the molecules.

Sodium dodecyl sulphate (SDS) is an anionic detergent which denatures proteins by “wrapping around” the polypeptide backbone—and SDS binds to proteins fairly specifically in a mass ratio of 1.4:1. In so doing, SDS confers a negative charge to the polypeptide in proportion to its length. Further, it is usually necessary to reduce disulphide bridges in proteins (denature) before they adopt the random-coil configuration necessary for separation by size; this is done with 2-mercaptoethanol or dithiothreitol (DTT). In denaturing SDS-PAGE separations therefore, migration is determined not by intrinsic electrical charge of the polypeptide, but by molecular weight.

Determination of molecular weight is done by SDS-PAGE of proteins of known molecular weight along with the protein to be characterized. A linear relationship exists between the logarithm of the molecular weight of an SDS-denatured polypeptide, or native nucleic acid, and its Rf. The Rf is calculated as the ratio of the distance migrated by the molecule to that migrated by a marker dye-front. A simple way of determining relative molecular weight by electrophoresis (Mr) is to plot a standard curve of distance migrated vs. log 10MW for known samples, and read off the log Mr of the sample after measuring distance migrated on the same gel.

In two-dimensional electrophoresis, proteins are fractionated first on the basis of one physical property, and, in a second step, on the basis of another. For example, isoelectric focusing can be used for the first dimension, conveniently carried out in a tube gel, and SDS electrophoresis in a slab gel can be used for the second dimension. One example of a procedure is that of O'Farrell, P. H., High Resolution Two-dimensional Electrophoresis of Proteins, J. Biol. Chem. 250:4007-4021 (1975), herein incorporated by reference in its entirety for its teaching regarding two-dimensional electrophoresis methods. Other examples include but are not limited to, those found in Anderson, L and Anderson, NG, High resolution two-dimensional electrophoresis of human plasma proteins, Proc. Natl. Acad. Sci. 74:5421-5425 (1977), Ornstein, L., Disc electrophoresis, L. Ann. N.Y. Acad. Sci. 121:321349 (1964), each of which is herein incorporated by reference in its entirety for teachings regarding electrophoresis methods. Laemmli, U.K., Cleavage of structural proteins during the assembly of the head of bacteriophage T4, Nature 227:680 (1970), which is herein incorporated by reference in its entirety for teachings regarding electrophoresis methods, discloses a discontinuous system for resolving proteins denatured with SDS. The leading ion in the Laemmli buffer system is chloride, and the trailing ion is glycine. Accordingly, the resolving gel and the stacking gel are made up in Tris-HCl buffers (of different concentration and pH), while the tank buffer is Tris-glycine. All buffers contain 0.1% SDS.

One example of an immunoassay that uses electrophoresis that is contemplated in the current methods is Western blot analysis. Western blotting or immunoblotting allows the determination of the molecular mass of a protein and the measurement of relative amounts of the protein present in different samples. Detection methods include chemiluminescence and chromagenic detection. Standard methods for Western blot analysis can be found in, for example, D. M. Bollag et al., Protein Methods (2d edition 1996) and E. Harlow & D. Lane, Antibodies, a Laboratory Manual (1988), U.S. Pat. No. 4,452,901, each of which is herein incorporated by reference in their entirety for teachings regarding Western blot methods. Generally, proteins are separated by gel electrophoresis, usually SDS-PAGE. The proteins are transferred to a sheet of special blotting paper, e.g., nitrocellulose, though other types of paper, or membranes, can be used. The proteins retain the same pattern of separation they had on the gel. The blot is incubated with a generic protein (such as milk proteins) to bind to any remaining sticky places on the nitrocellulose. An antibody is then added to the solution which is able to bind to its specific protein.

The attachment of specific antibodies to specific immobilized antigens can be readily visualized by indirect enzyme immunoassay techniques, usually using a chromogenic substrate (e.g. alkaline phosphatase or horseradish peroxidase) or chemiluminescent substrates. Other possibilities for probing include the use of fluorescent or radioisotope labels (e.g., fluorescein, ¹²⁵I). Probes for the detection of antibody binding can be conjugated anti-immunoglobulins, conjugated staphylococcal Protein A (binds IgG), or probes to biotinylated primary antibodies (e.g., conjugated avidin/streptavidin).

The power of the technique lies in the simultaneous detection of a specific protein by means of its antigenicity, and its molecular mass. Proteins are first separated by mass in the SDS-PAGE, then specifically detected in the immunoassay step. Thus, protein standards (ladders) can be run simultaneously in order to approximate molecular mass of the protein of interest in a heterogeneous sample.

The gel shift assay or electrophoretic mobility shift assay (EMSA) can be used to detect the interactions between DNA binding proteins and their cognate DNA recognition sequences, in both a qualitative and quantitative manner. Exemplary techniques are described in Ornstein L., Disc electrophoresis—I: Background and theory, Ann. NY Acad. Sci. 121:321-349 (1964), and Matsudiara, P T and D R Burgess, SDS microslab linear gradient polyacrylamide gel electrophoresis, Anal. Biochem. 87:386-396 (1987), each of which is herein incorporated by reference in its entirety for teachings regarding gel-shift assays.

In a general gel-shift assay, purified proteins or crude cell extracts can be incubated with a labeled (e.g., ³²P-radiolabeled) DNA or RNA probe, followed by separation of the complexes from the free probe through a nondenaturing polyacrylamide gel. The complexes migrate more slowly through the gel than unbound probe. Depending on the activity of the binding protein, a labeled probe can be either double-stranded or single-stranded. For the detection of DNA binding proteins such as transcription factors, either purified or partially purified proteins, or nuclear cell extracts can be used. For detection of RNA binding proteins, either purified or partially purified proteins, or nuclear or cytoplasmic cell extracts can be used. The specificity of the DNA or RNA binding protein for the putative binding site is established by competition experiments using DNA or RNA fragments or oligonucleotides containing a binding site for the protein of interest, or other unrelated sequence. The differences in the nature and intensity of the complex formed in the presence of specific and nonspecific competitor allows identification of specific interactions. Refer to Promega, Gel Shift Assay FAQ, available at <http://www.promega.com/faq/gelshfaq.html> (last visited Mar. 25, 2005), which is herein incorporated by reference in its entirety for teachings regarding gel shift methods.

Gel shift methods can include using, for example, colloidal forms of COOMASSIE (Imperial Chemicals Industries, Ltd) blue stain to detect proteins in gels such as polyacrylamide electrophoresis gels. Such methods are described, for example, in Neuhoff et al., Electrophoresis 6:427-448 (1985), and Neuhoff et al., Electrophoresis 9:255-262 (1988), each of which is herein incorporated by reference in its entirety for teachings regarding gel shift methods. In addition to the conventional protein assay methods referenced above, a combination cleaning and protein staining composition is described in U.S. Pat. No. 5,424,000, herein incorporated by reference in its entirety for its teaching regarding gel shift methods. The solutions can include phosphoric, sulfuric, and nitric acids, and Acid Violet dye.

Radioimmune Precipitation Assay (RIPA) is a sensitive assay using radiolabeled antigens to detect specific antibodies in serum. The antigens are allowed to react with the serum and then precipitated using a special reagent such as, for example, protein A sepharose beads. The bound radiolabeled immunoprecipitate is then commonly analyzed by gel electrophoresis. Radioimmunoprecipitation assay (RIPA) is often used as a confirmatory test for diagnosing the presence of HIV antibodies. RIPA is also referred to in the art as Farr Assay, Precipitin Assay, Radioimmune Precipitin Assay; Radioimmunoprecipitation Analysis; Radioimmunoprecipitation Analysis, and Radioimmunoprecipitation Analysis.

While the above immunoassays that utilize electrophoresis to separate and detect the specific proteins of interest allow for evaluation of protein size, they are not very sensitive for evaluating protein concentration. However, also contemplated are immunoassays wherein the protein or antibody specific for the protein is bound to a solid support (e.g., tube, well, bead, or cell) to capture the antibody or protein of interest, respectively, from a sample, combined with a method of detecting the protein or antibody specific for the protein on the support. Examples of such immunoassays include Radioimmunoassay (RIA), Enzyme-Linked Immunosorbent Assay (ELISA), Flow cytometry, protein array, multiplexed bead assay, and magnetic capture.

Radioimmunoassay (RIA) is a classic quantitative assay for detection of antigen-antibody reactions using a radioactively labeled substance (radioligand), either directly or indirectly, to measure the binding of the unlabeled substance to a specific antibody or other receptor system. Radioimmunoassay is used, for example, to test hormone levels in the blood without the need to use a bioassay. Non-immunogenic substances (e.g., haptens) can also be measured if coupled to larger carrier proteins (e.g., bovine gamma-globulin or human serum albumin) capable of inducing antibody formation. RIA involves mixing a radioactive antigen (because of the ease with which iodine atoms can be introduced into tyrosine residues in a protein, the radioactive isotopes ¹²⁵I or ¹³¹I are often used) with antibody to that antigen. The antibody is generally linked to a solid support, such as a tube or beads. Unlabeled or “cold” antigen is then adding in known quantities and measuring the amount of labeled antigen displaced. Initially, the radioactive antigen is bound to the antibodies. When cold antigen is added, the two compete for antibody binding sites—and at higher concentrations of cold antigen, more binds to the antibody, displacing the radioactive variant. The bound antigens are separated from the unbound ones in solution and the radioactivity of each used to plot a binding curve. The technique is both extremely sensitive, and specific.

Enzyme-Linked Immunosorbent Assay (ELISA), or more generically termed EIA (Enzyme ImmunoAssay), is an immunoassay that can detect an antibody specific for a protein. In such an assay, a detectable label bound to either an antibody-binding or antigen-binding reagent is an enzyme. When exposed to its substrate, this enzyme reacts in such a manner as to produce a chemical moiety which can be detected, for example, by spectrophotometric, fluorometric or visual means. Enzymes which can be used to detectably label reagents useful for detection include, but are not limited to, horseradish peroxidase, alkaline phosphatase, glucose oxidase, β-galactosidase, ribonuclease, urease, catalase, malate dehydrogenase, staphylococcal nuclease, asparaginase, yeast alcohol dehydrogenase, alpha.-glycerophosphate dehydrogenase, triose phosphate isomerase, glucose-6-phosphate dehydrogenase, glucoamylase and acetylcholinesterase.

Variations of ELISA techniques are know to those of skill in the art. In one variation, antibodies that can bind to proteins can be immobilized onto a selected surface exhibiting protein affinity, such as a well in a polystyrene microtiter plate. Then, a test composition suspected of containing a marker antigen can be added to the wells. After binding and washing to remove non-specifically bound immunocomplexes, the bound antigen can be detected. Detection can be achieved by the addition of a second antibody specific for the target protein, which is linked to a detectable label. This type of ELISA is a simple “sandwich ELISA.” Detection also can be achieved by the addition of a second antibody, followed by the addition of a third antibody that has binding affinity for the second antibody, with the third antibody being linked to a detectable label.

Another variation is a competition ELISA. In competition ELISA's, test samples compete for binding with known amounts of labeled antigens or antibodies. The amount of reactive species in the sample can be determined by mixing the sample with the known labeled species before or during incubation with coated wells. The presence of reactive species in the sample acts to reduce the amount of labeled species available for binding to the well and thus reduces the ultimate signal.

Regardless of the format employed, ELISAs have certain features in common, such as coating, incubating or binding, washing to remove non-specifically bound species, and detecting the bound immunecomplexes. Antigen or antibodies can be linked to a solid support, such as in the form of plate, beads, dipstick, membrane or column matrix, and the sample to be analyzed applied to the immobilized antigen or antibody. In coating a plate with either antigen or antibody, one will generally incubate the wells of the plate with a solution of the antigen or antibody, either overnight or for a specified period of hours. The wells of the plate can then be washed to remove incompletely adsorbed material. Any remaining available surfaces of the wells can then be “coated” with a nonspecific protein that is antigenically neutral with regard to the test antisera. These include bovine serum albumin (BSA), casein and solutions of milk powder. The coating allows for blocking of nonspecific adsorption sites on the immobilizing surface and thus reduces the background caused by nonspecific binding of antisera onto the surface.

In ELISAs, a secondary or tertiary detection means rather than a direct procedure can also be used. Thus, after binding of a protein or antibody to the well, coating with a non-reactive material to reduce background, and washing to remove unbound material, the immobilizing surface is contacted with the control clinical or biological sample to be tested under conditions effective to allow immunecomplex (antigen/antibody) formation. Detection of the immunecomplex then requires a labeled secondary binding agent or a secondary binding agent in conjunction with a labeled third binding agent.

Enzyme-Linked Immunospot Assay (ELISPOT) is an immunoassay that can detect an antibody specific for a protein or antigen. In such an assay, a detectable label bound to either an antibody-binding or antigen-binding reagent is an enzyme. When exposed to its substrate, this enzyme reacts in such a manner as to produce a chemical moiety which can be detected, for example, by spectrophotometric, fluorometric or visual means. Enzymes which can be used to detectably label reagents useful for detection include, but are not limited to, horseradish peroxidase, alkaline phosphatase, glucose oxidase, 0-galactosidase, ribonuclease, urease, catalase, malate dehydrogenase, staphylococcal nuclease, asparaginase, yeast alcohol dehydrogenase, alpha.-glycerophosphate dehydrogenase, triose phosphate isomerase, glucose-6-phosphate dehydrogenase, glucoamylase and acetylcholinesterase. In this assay a nitrocellulose microtiter plate is coated with antigen. The test sample is exposed to the antigen and then reacted similarly to an ELISA assay. Detection differs from a traditional ELISA in that detection is determined by the enumeration of spots on the nitrocellulose plate. The presence of a spot indicates that the sample reacted to the antigen. The spots can be counted and the number of cells in the sample specific for the antigen determined.

“Under conditions effective to allow immunecomplex (antigen/antibody) formation” means that the conditions include diluting the antigens and antibodies with solutions such as BSA, bovine gamma globulin (BGG) and phosphate buffered saline (PBS)/Tween so as to reduce non-specific binding and to promote a reasonable signal to noise ratio.

The suitable conditions also mean that the incubation is at a temperature and for a period of time sufficient to allow effective binding. Incubation steps can typically be from about 1 minute to twelve hours, at temperatures of about 20° to 30° C., or can be incubated overnight at about 0° C. to about 10° C.

Following all incubation steps in an ELISA, the contacted surface can be washed so as to remove non-complexed material. A washing procedure can include washing with a solution such as PBS/Tween or borate buffer. Following the formation of specific immunecomplexes between the test sample and the originally bound material, and subsequent washing, the occurrence of even minute amounts of immunecomplexes can be determined.

To provide a detecting means, the second or third antibody can have an associated label to allow detection, as described above. This can be an enzyme that can generate color development upon incubating with an appropriate chromogenic substrate. Thus, for example, one can contact and incubate the first or second immunecomplex with a labeled antibody for a period of time and under conditions that favor the development of further immunecomplex formation (e.g., incubation for 2 hours at room temperature in a PBS-containing solution such as PBS-Tween).

After incubation with the labeled antibody, and subsequent to washing to remove unbound material, the amount of label can be quantified, e.g., by incubation with a chromogenic substrate such as urea and bromocresol purple or 2,2′-azido-di-(3-ethyl-benzthiazoline-6-sulfonic acid [ABTS] and H₂O₂, in the case of peroxidase as the enzyme label. Quantitation can then be achieved by measuring the degree of color generation, e.g., using a visible spectra spectrophotometer.

Protein arrays are solid-phase ligand binding assay systems using immobilized proteins on surfaces which include glass, membranes, microtiter wells, mass spectrometer plates, and beads or other particles. The assays are highly parallel (multiplexed) and often miniaturized (microarrays, protein chips). Their advantages include being rapid and automatable, capable of high sensitivity, economical on reagents, and giving an abundance of data for a single experiment. Bioinformatics support is important; the data handling demands sophisticated software and data comparison analysis. However, the software can be adapted from that used for DNA arrays, as can much of the hardware and detection systems.

One of the chief formats is the capture array, in which ligand-binding reagents, which are usually antibodies but can also be alternative protein scaffolds, peptides or nucleic acid aptamers, are used to detect target molecules in mixtures such as plasma or tissue extracts. In diagnostics, capture arrays can be used to carry out multiple immunoassays in parallel, both testing for several analytes in individual sera for example and testing many serum samples simultaneously. In proteomics, capture arrays are used to quantitate and compare the levels of proteins in different samples in health and disease, i.e. protein expression profiling. Proteins other than specific ligand binders are used in the array format for in vitro functional interaction screens such as protein-protein, protein-DNA, protein-drug, receptor-ligand, enzyme-substrate, etc. The capture reagents themselves are selected and screened against many proteins, which can also be done in a multiplex array format against multiple protein targets.

For construction of arrays, sources of proteins include cell-based expression systems for recombinant proteins, purification from natural sources, production in vitro by cell-free translation systems, and synthetic methods for peptides. Many of these methods can be automated for high throughput production. For capture arrays and protein function analysis, it is important that proteins should be correctly folded and functional; this is not always the case, e.g. where recombinant proteins are extracted from bacteria under denaturing conditions. Nevertheless, arrays of denatured proteins are useful in screening antibodies for cross-reactivity, identifying autoantibodies and selecting ligand binding proteins.

Protein arrays have been designed as a miniaturization of familiar immunoassay methods such as ELISA and dot blotting, often utilizing fluorescent readout, and facilitated by robotics and high throughput detection systems to enable multiple assays to be carried out in parallel. Commonly used physical supports include glass slides, silicon, microwells, nitrocellulose or PVDF membranes, and magnetic and other microbeads. While microdrops of protein delivered onto planar surfaces are the most familiar format, alternative architectures include CD centrifugation devices based on developments in microfluidics (Gyros, Monmouth Junction, NJ) and specialised chip designs, such as engineered microchannels in a plate (e.g., The Living Chip™, Biotrove, Woburn, MA) and tiny 3D posts on a silicon surface (Zyomyx, Hayward CA). Particles in suspension can also be used as the basis of arrays, providing they are coded for identification; systems include colour coding for microbeads (Luminex, Austin, TX; Bio-Rad Laboratories) and semiconductor nanocrystals (e.g., QDots™, Quantum Dot, Hayward, CA), and barcoding for beads (UltraPlex™, SmartBead Technologies Ltd, Babraham, Cambridge, UK) and multimetal microrods (e.g., Nanobarcodes™ particles, Nanoplex Technologies, Mountain View, CA). Beads can also be assembled into planar arrays on semiconductor chips (LEAPS technology, BioArray Solutions, Warren, NJ).

Immobilization of proteins involves both the coupling reagent and the nature of the surface being coupled to. A good protein array support surface is chemically stable before and after the coupling procedures, allows good spot morphology, displays minimal nonspecific binding, does not contribute a background in detection systems, and is compatible with different detection systems. The immobilization method used are reproducible, applicable to proteins of different properties (size, hydrophilic, hydrophobic), amenable to high throughput and automation, and compatible with retention of fully functional protein activity. Orientation of the surface-bound protein is recognized as an important factor in presenting it to ligand or substrate in an active state; for capture arrays the most efficient binding results are obtained with orientated capture reagents, which generally require site-specific labeling of the protein.

Both covalent and noncovalent methods of protein immobilization are used and have various pros and cons. Passive adsorption to surfaces is methodologically simple, but allows little quantitative or orientational control; it may or may not alter the functional properties of the protein, and reproducibility and efficiency are variable. Covalent coupling methods provide a stable linkage, can be applied to a range of proteins and have good reproducibility; however, orientation may be variable, chemical derivatization may alter the function of the protein and requires a stable interactive surface. Biological capture methods utilizing a tag on the protein provide a stable linkage and bind the protein specifically and in reproducible orientation, but the biological reagent must first be immobilized adequately and the array may require special handling and have variable stability.

Several immobilization chemistries and tags have been described for fabrication of protein arrays. Substrates for covalent attachment include glass slides coated with amino- or aldehyde-containing silane reagents. In the Versalinx™ system (Prolinx, Bothell, WA) reversible covalent coupling is achieved by interaction between the protein derivatised with phenyldiboronic acid, and salicylhydroxamic acid immobilized on the support surface. This also has low background binding and low intrinsic fluorescence and allows the immobilized proteins to retain function. Noncovalent binding of unmodified protein occurs within porous structures such as HydroGel™ (PerkinElmer, Wellesley, MA), based on a 3-dimensional polyacrylamide gel; this substrate is reported to give a particularly low background on glass microarrays, with a high capacity and retention of protein function. Widely used biological coupling methods are through biotin/streptavidin or hexahistidine/Ni interactions, having modified the protein appropriately. Biotin may be conjugated to a poly-lysine backbone immobilised on a surface such as titanium dioxide (Zyomyx) or tantalum pentoxide (Zeptosens, Witterswil, Switzerland).

Array fabrication methods include robotic contact printing, ink-jetting, piezoelectric spotting and photolithography. A number of commercial arrayers are available [e.g. Packard Biosciences] as well as manual equipment [V & P Scientific]. Bacterial colonies can be robotically gridded onto PVDF membranes for induction of protein expression in situ.

At the limit of spot size and density are nanoarrays, with spots on the nanometer spatial scale, enabling thousands of reactions to be performed on a single chip less than 1 mm square. BioForce Laboratories have developed nanoarrays with 1521 protein spots in 85sq microns, equivalent to 25 million spots per sq cm, at the limit for optical detection; their readout methods are fluorescence and atomic force microscopy (AFM).

Fluorescence labeling and detection methods are widely used. The same instrumentation as used for reading DNA microarrays is applicable to protein arrays. For differential display, capture (e.g., antibody) arrays can be probed with fluorescently labeled proteins from two different cell states, in which cell lysates are directly conjugated with different fluorophores (e.g. Cy-3, Cy-5) and mixed, such that the color acts as a readout for changes in target abundance. Fluorescent readout sensitivity can be amplified 10-100 fold by tyramide signal amplification (TSA) (PerkinElmer Lifesciences). Planar waveguide technology (Zeptosens) enables ultrasensitive fluorescence detection, with the additional advantage of no intervening washing procedures. High sensitivity can also be achieved with suspension beads and particles, using phycoerythrin as label (Luminex) or the properties of semiconductor nanocrystals (Quantum Dot). A number of novel alternative readouts have been developed, especially in the commercial biotech arena. These include adaptations of surface plasmon resonance (HTS Biosystems, Intrinsic Bioprobes, Tempe, AZ), rolling circle DNA amplification (Molecular Staging, New Haven CT), mass spectrometry (Intrinsic Bioprobes; Ciphergen, Fremont, CA), resonance light scattering (Genicon Sciences, San Diego, CA) and atomic force microscopy [BioForce Laboratories].

Capture arrays form the basis of diagnostic chips and arrays for expression profiling. They employ high affinity capture reagents, such as conventional antibodies, single domains, engineered scaffolds, peptides or nucleic acid aptamers, to bind and detect specific target ligands in high throughput manner.

Antibody arrays have the required properties of specificity and acceptable background, and some are available commercially (BD Biosciences, San Jose, CA; Clontech, Mountain View, CA; BioRad; Sigma, St. Louis, MO). Antibodies for capture arrays are made either by conventional immunization (polyclonal sera and hybridomas), or as recombinant fragments, usually expressed in E. coli, after selection from phage or ribosome display libraries (Cambridge Antibody Technology, Cambridge, UK; BioInvent, Lund, Sweden; Affitech, Walnut Creek, CA; Biosite, San Diego, CA). In addition to the conventional antibodies, Fab and scFv fragments, single V-domains from camelids or engineered human equivalents (Domantis, Waltham, MA) may also be useful in arrays.

The term “scaffold” refers to ligand-binding domains of proteins, which are engineered into multiple variants capable of binding diverse target molecules with antibody-like properties of specificity and affinity. The variants can be produced in a genetic library format and selected against individual targets by phage, bacterial or ribosome display. Such ligand-binding scaffolds or frameworks include ‘Affibodies’ based on Staph. aureus protein A (Affibody, Bromma, Sweden), ‘Trinectins’ based on fibronectins (Phylos, Lexington, MA) and ‘Anticalins’ based on the lipocalin structure (Pieris Proteolab, Freising-Weihenstephan, Germany). These can be used on capture arrays in a similar fashion to antibodies and may have advantages of robustness and ease of production.

Nonprotein capture molecules, notably the single-stranded nucleic acid aptamers which bind protein ligands with high specificity and affinity, are also used in arrays (SomaLogic, Boulder, CO). Aptamers are selected from libraries of oligonucleotides by the Selex™ procedure and their interaction with protein can be enhanced by covalent attachment, through incorporation of brominated deoxyuridine and UV-activated crosslinking (photoaptamers). Photocrosslinking to ligand reduces the crossreactivity of aptamers due to the specific steric requirements. Aptamers have the advantages of ease of production by automated oligonucleotide synthesis and the stability and robustness of DNA; on photoaptamer arrays, universal fluorescent protein stains can be used to detect binding.

Protein analytes binding to antibody arrays may be detected directly or via a secondary antibody in a sandwich assay. Direct labelling is used for comparison of different samples with different colours. Where pairs of antibodies directed at the same protein ligand are available, sandwich immunoassays provide high specificity and sensitivity and are therefore the method of choice for low abundance proteins such as cytokines; they also give the possibility of detection of protein modifications. Label-free detection methods, including mass spectrometry, surface plasmon resonance and atomic force microscopy, avoid alteration of ligand. What is required from any method is optimal sensitivity and specificity, with low background to give high signal to noise. Since analyte concentrations cover a wide range, sensitivity has to be tailored appropriately; serial dilution of the sample or use of antibodies of different affinities are solutions to this problem. Proteins of interest are frequently those in low concentration in body fluids and extracts, requiring detection in the pg range or lower, such as cytokines or the low expression products in cells.

An alternative to an array of capture molecules is one made through ‘molecular imprinting’ technology, in which peptides (e.g., from the C-terminal regions of proteins) are used as templates to generate structurally complementary, sequence-specific cavities in a polymerizable matrix; the cavities can then specifically capture (denatured) proteins that have the appropriate primary amino acid sequence (ProteinPrint™, Aspira Biosystems, Burlingame, CA).

Another methodology which can be used diagnostically and in expression profiling is the ProteinChip® array (Ciphergen, Fremont, CA), in which solid phase chromatographic surfaces bind proteins with similar characteristics of charge or hydrophobicity from mixtures such as plasma or tumour extracts, and SELDI-TOF mass spectrometry is used to detection the retained proteins.

Large-scale functional chips have been constructed by immobilizing large numbers of purified proteins and used to assay a wide range of biochemical functions, such as protein interactions with other proteins, drug-target interactions, enzyme-substrates, etc. Generally they require an expression library, cloned into E. coli, yeast or similar from which the expressed proteins are then purified, e.g. via a His tag, and immobilized. Cell free protein transcription/translation is a viable alternative for synthesis of proteins which do not express well in bacterial or other in vivo systems.

For detecting protein-protein interactions, protein arrays can be in vitro alternatives to the cell-based yeast two-hybrid system and may be useful where the latter is deficient, such as interactions involving secreted proteins or proteins with disulphide bridges. High-throughput analysis of biochemical activities on arrays has been described for yeast protein kinases and for various functions (protein-protein and protein-lipid interactions) of the yeast proteome, where a large proportion of all yeast open-reading frames was expressed and immobilised on a microarray. Large-scale ‘proteome chips’ promise to be very useful in identification of functional interactions, drug screening, etc. (Proteometrix, Branford, CT).

As a two-dimensional display of individual elements, a protein array can be used to screen phage or ribosome display libraries, in order to select specific binding partners, including antibodies, synthetic scaffolds, peptides and aptamers. In this way, ‘library against library’ screening can be carried out. Screening of drug candidates in combinatorial chemical libraries against an array of protein targets identified from genome projects is another application of the approach.

A multiplexed bead assay, such as, for example, the BD™ Cytometric Bead Array, is a series of spectrally discrete particles that can be used to capture and quantitate soluble analytes. The analyte is then measured by detection of a fluorescence-based emission and flow cytometric analysis. Multiplexed bead assay generates data that is comparable to ELISA based assays, but in a “multiplexed” or simultaneous fashion. Concentration of unknowns is calculated for the cytometric bead array as with any sandwich format assay, i.e. through the use of known standards and plotting unknowns against a standard curve. Further, multiplexed bead assay allows quantification of soluble analytes in samples never previously considered due to sample volume limitations. In addition to the quantitative data, powerful visual images can be generated revealing unique profiles or signatures that provide the user with additional information at a glance.

### D. EXAMPLES

The following examples are put forth so as to provide those of ordinary skill in the art with a complete disclosure and description of how the compounds, compositions, articles, devices and/or methods claimed herein are made and evaluated, and are intended to be purely exemplary and are not intended to limit the disclosure. Efforts have been made to ensure accuracy with respect to numbers (e.g., amounts, temperature, etc.), but some errors and deviations should be accounted for. Unless indicated otherwise, parts are parts by weight, temperature is in ° C. or is at ambient temperature, and pressure is at or near atmospheric.

### Example 1: Identification of a Biomarker Panel from Genome-Wide Methylation to Detect Early HPV-Associated Oropharyngeal Cancer

### a) Methods

### (1) Study Population

Men, 18 years of age and older, recently diagnosed with a new histologically confirmed OPC (C01.9 base of tongue; C05.1 soft palate, not otherwise specified [NOS]; C05.2 uvula; C09.0 tonsillar fossa; C09.1 tonsillar pillar; C09.8 overlapping lesion of the tonsil; C09.9 tonsil, NOS; C10.0 vallecula; C10.2 lateral wall of epiglottis; C10.3 posterior wall of epiglottis; C10.8 overlapping lesion of oropharynx and C10.9 oropharynx, NOS.) were recruited from May 2014 to March 2020 from the Moffitt Cancer Center Head and Neck Cancer Radiation Oncology and Senior Adult Oncology clinics. Patients were pre-screened for eligibility by reviewing medical records. Interested and eligible cases signed an informed consent. Cases who had received treatment prior to enrollment or did not complete the study survey were excluded.

Approval was obtained from Advarra Institutional Review Board and the Moffitt Cancer Center Scientific Review Committee. Cases were defined as either early (T1-T2, N0-N1; small tumors with only a single ipsilateral positive node <3 cm) or late disease 116. Healthy, cancer-free controls were selected from the US participants of the HPV Infection in Men (HIM) Study which recruited men aged 18-70 years, with no prior history of an HPV-related cancers, HPV vaccination, or HIV/AIDS. Controls were frequency matched on age within 5 years and smoking history (never, former, current).

In both studies, demographic information as well as sexual behavior and substance use history was collected via computer-assisted risk survey during the study visit. Men then provided an oral gargle sample by gargling mouthwash for 30 seconds then dispensing it into a 50 ml conical tube. For processing, specimens were centrifuged at 2,000 g for 15 min, then the cell pellet underwent three wash procedures by resuspending in 20 ml cold phosphate buffered solution (PBS), repeatedly mixed by inversion to assure thorough homogenization, then centrifuged at 2,000 g for 15 min at 4° C. The remaining cell pellet was resuspended in 1.2 ml PBS and archived at −80° C. until analysis. Oral HPV DNA was extracted from oral gargle cell pellets using the automated BioRobot MDx (Qiagen). HPV status of oral gargle specimens for all participants was obtained using the HPV SPF₁₀ PCR-DEIA-LiPA₂₅ line probe assay (DDL Diagnostic Laboratory, Rijswik, the Netherlands).

### (2) Methylation Studies

### (a) HPV and Host Gene Methylation.

All oral gargle samples were tested for methylation of three CpG sites (438, 427 and 425) in the host tumor suppressor gene EPB41L3, using a validated pyrosequencing method (PyroMark). Briefly, 200 ng of DNA was used in bisulfite conversion reactions in which unmethylated cytosines were converted to uracil using the EZ DNA methylation kit (Zymo Research, Irvine, CA). Converted DNA was purified and amplified by PCR primers, with one biotin primer for each pair. Primers were designed with short amplicons (90-140 base pairs each) using the PyroMark Assay Design software (V2.0.1.15 Qiagen). PCR was performed using a converted DNA equivalent of 1,500 cells using the Pyro-Mark PCR kit (Qiagen). PCR products were then captured by streptavidin beads (GE Healthcare, Buckinghamshire, UK) in 96-well plates and pyrosequenced using PyroGold reagents with the signal analyzed using a PyroMark TMQ96 ID (Qiagen) instrument. All runs included standard curves as positive controls of 0, 50 and 100% methylated human DNA and a non-template control.

### (b) Genome-Wide Methylation.

Extracted DNA was also processed for genome-wide methylation using the MethylationEPIC BeadChip (Illumina) array which evaluates methylation status of >850,000 CpG sites across the genome. 500 ng of extracted and Qubit-quantitated DNA was bisulfite converted using Zymo EZ DNA Methylation Kits (Zymo Research, Irvine, CA) and the converted DNA was used to process and hybridize the Illumina MethylationEPIC BeadChip according to the manufacturer's protocol (Illumina, Inc., San Diego, CA). The single-base extension and staining were performed using the Tecan Evo liquid handling system with the Te-Flow (GenePaint) chamber. The arrays were then scanned on an Illumina iScan scanner, and QC was performed using the Illumina GenomeStudio software. Utilizing the minfi R package, the resulting IDAT files were subsequently read and parsed, and then the raw intensity values were preprocessed with the Funnorm method using the preprocess Funnorm function. Quality control consisted of plotting and inspecting the sample-level histograms of beta values and of performing a principal components analysis (PCA). One sample failed based on its beta histogram and the fact that it was clearly an outlier in the plot of principal component 3 against 4. Removal of that sample resulted in a data set consisting of 89 early OPC cases and 108 control cases. Beta values were calculated as the ratio of methylation to total signal intensity (methylated signal plus unmethylated signal). Any beta value that showed a detection p-value>0.01 was marked as NA, and then a probe was filtered from further analysis if that probe (1) had a missing value in 20% or more of the samples or (2) had a range less than 0.1 across all samples.

### (3) Building a Biomarker Panel

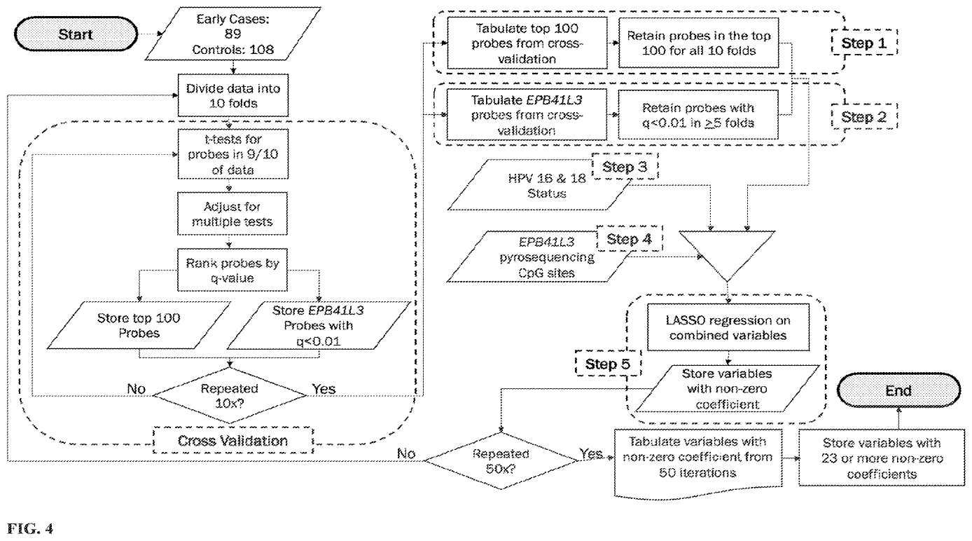

Data from the methylation studies were analyzed to build a biomarker panel capable of distinguishing early OPC from controls. The overall process consisted of five additive steps: 1) identify candidate Illumina probes from all available probes; 2) use alternative selection criteria to find key candidate Illumina probes from the EPB41L3 gene which is known to be associated with HPV-associated cervical pre-cancer and HPV-OPC; 3) manually add the methylation data from EPB41L3 pyrosequencing CpGs 4) manually add the HPV16 and HPV18 status and 5) build a LASSO model using the variables identified in the previous steps to determine which variables are given a non-zero coefficient. The steps towards building a biomarker panel are further described herein and in FIG. 4.

Steps 1 and 2 consisted of using ten-fold cross-validation to independently compare detection at individual CpG sites to identify the Illumina probes that were differentially methylated between early OPC cases and controls. First, t-tests were systematically performed on 9 out of the 10 sets, so that one set was left out each time. This resulted in 10 rounds of ~850,000 t-tests. The p-values from the t-tests were transformed into q-values to control for the multiple testing. The top 100 probes (lowest q values) and any EPB41L3 probe with a q-value less than 0.01 were stored from each round. After completion of all ten rounds of the cross-validation, probes in the top 100 for all rounds of the cross-validation and EPB41L3 probes that were selected at least 5 times out of the ten rounds were chosen to be candidates for further consideration.

The HPV16 and HPV18 status as binary categorical variables were added (step 3). Then candidate probes from the prior pyrosequencing methylation data, which included three CpG sites (438, 427 and 425) in the EPB41L3 gene (step 4) were added. It should be noted that these CpG sites are not present in the Illumina array. Finally in step 5, a logistic regression model with LASSO regularization was built with the glmnet (version 4.1-1) R package using the previously selected candidates, and those that were given a non-zero coefficient were stored.

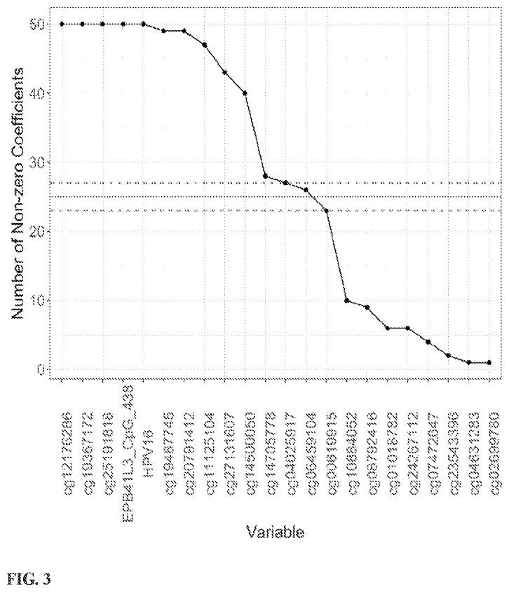

The above 5 steps were repeated for a total of 50 iterations. The candidate markers discovered at the end of each iteration were stored. The final panel of 14 variables were those that were given a non-zero coefficient in at least 23 out of the 50 iterations. There was a steep drop-off after 23 in the number of iterations a CpG site had a non-zero coefficient (FIG. 3). The final model was built as a simple logistic regression model consisting of those 14 selected variables. To estimate the accuracy of this final model on unseen data a bootstrap resampling method was employed in which a logistic regression model was trained on the bootstrap sample with the 14 selected variables and validated samples left out in each re-sampling set.

### (4) Statistical Analyses

Sociodemographic characteristics, sexual behavior, oral health, and oral gargle HPV status were compared for all cases (n=228) compared to controls (n=142) using the Cochran-Mantel-Haenszel (CMH) test. Models representing various steps in the probe selection process were compared using boxplots and AUCs were compared using Wilcoxon rank sum test. ROC curves from the training set and test set (from bootstrap re-sampling) were produced by varying the threshold across the predicted probabilities with area under the curve (AUC) generated by the Wilcoxon test. Cut-points were identified using Youden's J (J statistic) and were selected with a goal of maximizing specificity while maintaining a sensitivity greater than 70%. Steps to build the panel were completed with R. Other analyses were performed in SAS 9.3.

### b) Results

There were 228 OPC cases, 92 of which were identified as early OPC, and 142 age- and smoking-matched controls included in this study. Cases differed from controls significantly (p<0.05) by race, ethnicity, marital status, education, pack-years of smoking, kissing and oral sex history, tonsillectomy history, number of teeth extracted, and oral HPV infection with any type or HPV16. While matched on smoking status, cases were more likely to have higher total pack-years than controls (21% vs 13% of controls). Among cases, 68% had any HPV detected in the oral gargle, compared to 14% of controls; and 54% were positive for HPV16 compared to 3.5% of controls. (Table 1).

TABLE 1
Sociodemographic Characteristics of OPC Cases compared
to Controls

| | Control | Case | |
|---|---|---|---|
| Characteristics | N = 142¹ | N = 228¹ | p-value² |
| Race | | | 0.0002 |
| White | 116 (81.7%) | 216 (94.7%) | |
| Black | 21 (14.8%) | 7 (3.1%) | |
| Other | 4 (2.8%) | 5 (2.2%) | |
| N/A or refused | 1 (0.7%) | 0 (0%) | |
| Ethnicity | | | 0.0226 |
| Hispanic | 16 (11.3%) | 9 (3.9%) | |
| Non-Hispanic | 125 (88.0%) | 218 (95.6%) | |
| N/A or refused | 1 (0.7%) | 1 (0.4%) | |
| Median Age (years) (SD) | 59 (10) | 62 (10) | 0.3109 |
| Age (years) | | | 0.1168 |
| 35-49 | 22 (15.5%) | 22 (9.6%) | |
| 50-59 | 50 (35.2%) | 70 (30.7%) | |
| 60-69 | 44 (31.0%) | 77 (33.8%) | |
| >=70 | 25 (17.6%) | 59 (25.9%) | |
| Marital status | | | <0.0001 |
| Married/cohabiting | 80 (56.3%) | 181 (79.4%) | |
| Single/divorced/separated/ | 62 (43.7%) | 46 (20.2%) | |
| widowed | | | |
| N/A or refused | 0 (0%) | 1 (0.4%) | |
| Education | | | 0.0508 |
| High school (<12 years) | 17 (12.0%) | 54 (23.7%) | |
| Some college/vocational | 56 (39.4%) | 80 (35.1%) | |
| school | | | |
| College graduate | 41 (28.9%) | 62 (27.2%) | |
| Postgraduate/professional | 28 (19.7%) | 31 (13.6%) | |
| school | | | |
| N/A or refused | 0 (0%) | 1 (0.4%) | |
| Smoking status | | | 0.2216 |
| Never | 57 (40.1%) | 103 (45.2%) | |
| Former | 65 (45.8%) | 106 (46%) | |
| Current | 20 (14.1%) | 18 (7.9%) | |
| N/A or refused | 0 (0%) | 1 (0.4%) | |
| Pack-years smoking | | | 0.0130 |
| Never | 57 (40.1%) | 106 (46.5%) | |
| <= 5 | 30 (21.1%) | 27 (11.8%) | |
| 6-29 | 37 (26.1%) | 46 (20.2%) | |
| >=30 | 18 (12.7%) | 49 (21.5%) | |
| N/A or refused | 0 (0%) | 0 (0%) | |
| Alcohol drinks per occasion | | | 0.3569 |
| in the past month | | | |
| None | 40 (28.2%) | 77 (33.8%) | |
| 1-4 | 83 (58.5%) | 122 (53.5%) | |
| >=5 | 18 (12.7%) | 26 (11.4%) | |
| N/A or refused | 0 (0%) | 3 (1.3%) | |
| Lifetime number of people | | | 0.0181 |
| kissed with tongue | | | |
| None | 14 (9.9%) | 8 (3.5%) | |
| 1-9 | 43 (30.3%) | 53 (23.2%) | |
| 10-24 | 33 (23.2%) | 62 (27.2%) | |
| 25-49 | 24 (16.9%) | 34 (14.9%) | |
| >=50 | 21 (14.8%) | 60 (26.3%) | |
| N/A or refused | 7 (4.9%) | 11 (4.8%) | |
| Gave oral sex in past 6 months | | | 0.0032 |
| No | 75 (52.8%) | 121 (53.1%) | |
| Yes | 66 (46.5%) | 87 (38.2%) | |
| N/A or refused | 1 (0.7%) | 20 (8.8%) | |
| Tonsillectomy | | | <0.0001 |
| No | 128 (90.1%) | 122 (53.5%) | |
| Yes | 14 (9.9%) | 105 (46.1%) | |
| N/A or refused | 0 (0%) | 1 (0.4%) | |
| Time since tonsillectomy | | | <0.0001 |
| (years ago) | | | |
| Never removed | 128 (90.1%) | 122 (53.5%) | |
| <2 years | 0 (0%) | 23 (10.1%) | |
| 2-29 years | 1 (0.7%) | 7 (3.1%) | |
| 30+ years | 13 (9.2%) | 74 (32.5%) | |
| Gingivitis | | | 0.1683 |
| No | 99 (69.7%) | 169 (74.1%) | |
| Yes | 38 (26.8%) | 57 (25.0%) | |
| N/A or refused | 5 (3.5%) | 2 (0.9%) | |
| Teeth extracted prior to | | | <0.0001 |
| diagnosis | | | |
| 0 | 77 (54.2%) | 50 (21.9%) | |
| 1-9 | 48 (33.8%) | 104 (45.6%) | |
| >=10 | 13 (9.2%) | 33 (14.5%) | |
| N/A or refused | 4 (2.8%) | 41 (18.0%) | |
| Any HPV (Oral) | | | |
| Positive | 19 (17.6%) | 53 (59.6%) | <0.0001 |
| Negative | 89 (82.4%) | 36 (40.4%) | |
| HPV 16 (oral) | | | |
| Positive | 4 (3.7%) | 38 (42.7%) | <0.0001 |
| Negative | 104 (96.3%) | 51 (57.3%) | |
| HPV 18 (Oral) | | | |
| Positive | 1 (0.9%) | 4 (4.5%) | 0.1139 |
| Negative | 107 (99.1%) | 85 (95.5%) | |
| Tumor Location | | | |
| Tonsil | | 117 (51.3%) | |
| Base of tongue (BOT) | | 103 (45.2%) | |
| Other oropharynx | | 8 (3.5%) | |
| Early or late disease | | | |
| presentation | | | |
| Early | | 92 (40.4%) | |
| Late | | 136 (59.6%) | |
| P16ink4a (IHC) | | | |
| Positive | | 198 (86.8%) | |
| Negative | | 25 (11%) | |
| Any HPV type (oral) | | | <0.0001 |
| Positive | 20 (14.1%) | 155 (68.0%) | |
| Negative | 122 (85.9%) | 73 (32.0%) | |
| HPV 16 (oral) | | | <0.0001 |
| Positive | 5 (3.5%) | 122 (53.5%) | |
| Negative | 137 (96.5%) | 106 (46.5%) | |
| HPV 18 (oral) | | | 0.0617 |
| Positive | 1 (0.7%) | 9 (3.9%) | |
| Negative | 141 (99.3%) | 219 (96.1%) | |
| Any HPV type (tumor) | | | |
| Positive | | 203 (89.0%) | |
| Negative | | 24 (10.5%) | |
| HPV 16 (tumor) | | | |
| Positive | | 187 (82.0%) | |
| Negative | | 40 (17.5%) | |
| HPV 18 (tumor) | | | |
| Positive | | 9 (4.0%) | |
| Negative | | 218 (96.0%) | |
| HR HPV (other than | | | |
| 16/18) (tumor) | | | |
| Positive | | 10 (4.4%) | |
| Negative | | 217 (95.6%) | |

¹n (%); Median (SD)
²Cochran-Mantel-Haenszel (Exact)

The biomarker selection process revealed 14 markers significantly associated (p<0.05) with early OPC, including one EPB41L3 CpG site from the Illumina array, one EPB41L3 CpG site (438) from pyrosequencing and oral HPV16/18 status. (Table 2 and Table 3)

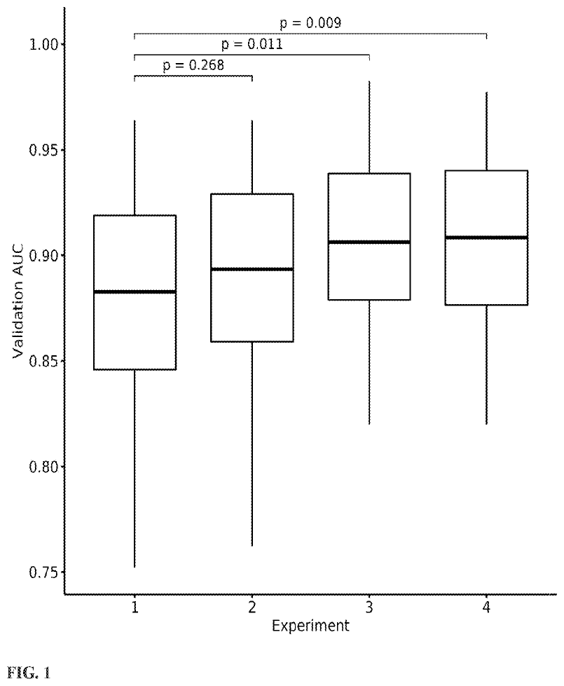

There was significant improvement (p=0.009) in the ability to predict early OPC compared to controls using the five-step process (AUC=0.908) described herein compared to a single-pass analysis of Illumina probes only (AUC=0.883). (FIG. 1).

TABLE 2
Description of 14 markers in the OPC biomarker panel

| Illumina | | | CpG Island | Methylation | |
|---|---|---|---|---|---|
| CpG | | Gene | (Relation to | | Mean |
| Probe | Chr^{a} | Name^{b} | CpG Island) | Change | Diff. |
| cg25191818 | 16 | PAPD5 | No | Increase | 0.018 |
| cg19367172 | 18 | ST8SIA5 | Yes | Increase | 0.045 |
| | | | (N_Shelf) | | |
| cg12176286 | 15 | SNORD115-20 | No | Increase | 0.063 |
| cg19487745 | 10 | Unknown | No | Increase | 0.034 |
| cg20791412 | 10 | Unknown | No | Decrease | −0.062 |
| cg11125104 | 2 | NCRNA00164; | Yes | Increase | 0.070 |
| | | MIR663B | (Island) | | |
| cg27131607 | 17 | DDX42 | Yes | Increase | 0.070 |
| | | | (Island) | | |
| cg14500050 | 13 | RASA3 | Yes | Increase | 0.037 |
| | | | (Island) | | |
| cg14705778 | 4 | HMGB2 | Yes | Decrease | −0.046 |
| | | | (Island) | | |
| cg04025917 | 19 | ARMC6 | Yes | Increase | 0.071 |
| | | | (Island) | | |
| cg06459104 | 18 | EPB41L3 | No | Decrease | −0.052 |
| cg00619915 | 2 | Unknown | No | Increase | 0.069 |

Markers in the panel from prior studies
EPB41L3 CpG site 438
HPV16 categorical
ªChromosome
^{b}Multiple genes listed indicates the CpG site occurs in an area in which the genes overlap and share a common region of the genome; missing gene symbol indicates the CpG site occurs on a gene not yet described

TABLE 3
Probe sequences

| Probe | chromosome | position | strand | Forward_Sequence |
|---|---|---|---|---|
| cg19487745 | chr10 | 45567697 | + | CTTGATCTCTTGACCTCGTGATCCGCCTGCCTTGGC |
| | | | | CTTCCAAAGTGCTGGGATTACAGG[CG]TGAGCCAC |
| | | | | CGCGGCCGGCCATTTTTGTTGTTGTTGTTGTTGTTG |
| | | | | TTGTTGTTGTTGTTGA (SEQ ID NO: 1) |
| cg14705778 | chr4 | 174254825 | − | TTTGCCCCGCGGCTTGTTGGGGTCTCCTTTACCCAT |
| | | | | GTTGACAGATCCGCGTCCACCTGA[CG]GGGCCGA |
| | | | | GGGGGGAGAGGGGAAGCCGGAGGGTCGGCGCG |
| | | | | GAGCCCGCAGCTGCCAGGGCG (SEQ ID NO: 2) |
| cg27131607 | chr17 | 61851115 | − | AGGCGAGGTGGGGCTTTTGGGAAAATTACATAATC |
| | | | | CCCGCCCCTGGGCCGGAACTGCCCT[CG]CCTATCC |
| | | | | CAATCCTCCTACCTAGGATTCCGTCAAGTTGACGTC |
| | | | | ACACGGAACCGGTGCTC (SEQ ID NO: 3) |
| cg04025917 | chr19 | 19144813 | + | GCTGCCGAGGCCTGTCCCGCGCGGAGTGGGGAGT |
| | | | | GGGGGTGCCGCAGGGTCGGGCTGGCG[CG]ACCCT |
| | | | | CGGGTACGTGGTTTGGGGGGAAAAAAAAAATGAA |
| | | | | AGAATTTTACAGAAGCCGCCT (SEQ ID NO: 4) |
| cg25191818 | chr16 | 50186632 | + | GGCGGGGCACACGTAGCCGTGCAGGGTCTCGCGC |
| | | | | AGCCCGCGTCAGGACCCCGGGGCACA[CG]CTCGG |
| | | | | GCAGGGGGCGTGTCCCCACGCCACCAGAAAAAGT |
| | | | | GTTATAGGCTGTTTTTTTTTT (SEQ ID NO: 5) |
| cg11125104 | chr2 | 133015268 | − | GCACACCTCTCAGATCGCTAGAGAAGGCTTTCTCA |
| | | | | CCGAGGGTGGGTCACACTCCCCACA[CG]CCAGTCG |
| | | | | CCCCTCGTCGGGCCCGCAGAGGCGCTCAGGGACG |
| | | | | CCTGGGGAAGGGAGGGGGC (SEQ ID NO: 6) |
| cg19367172 | chr18 | 44333130 | − | TTCCACCTCACAGCAGTTGTATCCATTTCTTCATTGT |
| | | | | GTCTCTTCCTGTTACAATCCCTG[CG]CGCACACACA |
| | | | | TAGTCCTACCTTCAATTATTTATTCATTATCCAATAT |
| | | | | TTATTGAGAACAT (SEQ ID NO: 7) |
| cg14500050 | chr13 | 114852691 | + | TCCTCGCGGGGAAATCCCGGGGAAGTGCTGAGTC |
| | | | | TGGAGTATCGACTCCCTTCGTCCTCG[CG]GGGAAA |
| | | | | TCACGGGGAAGTGCTGAGTCTGGAGTATTTATTCC |
| | | | | CTTCGTCCTCGCGGGGAAA (SEQ ID NO: 8) |
| cg20791412 | chr10 | 45567485 | − | TTTTTTTTTTTGAGACAGAGTCTCACTCTGTTGCTTG |
| | | | | ACTGGAGTGCATTGGCATGATCT[CG]GCTCACTGC |
| | | | | AACCTCTGCTTCCCGGGTTCAAGCGATTCTCCTGCC |
| | | | | TCAGCCTCCTGAGTA (SEQ ID NO: 9) |
| cg06459104 | chr18 | 5456880 | + | GACAGGGGTAAGGAGGAAGCTGCTGAACCCTGCT |
| | | | | AAGAAGATGGGAGGCCTGGTCTAATT[CG]AGAAC |
| | | | | CAGACACGGCAGGCCCTGTCTTCTGGGCTCAGCCC |
| | | | | AGTGAGAAGCTGGAGCAGCA (SEQ ID NO: 10) |
| cg00619915 | chr2 | 44497795 | + | TCATCCGACACCAGCCGCCTCCACCATGCCGCCGA |
| | | | | AGTTCCACCCCAATGAGATCAAAGT[CG]TATACTTC |
| | | | | ACGTGCACTGGAGTTGAAGTCGGTGCCACTTCTGC |
| | | | | TCTGGCCCCCAAGATTG (SEQ ID NO: 11) |
| cg12176286 | chr15 | 25452149 | + | GGGGCACTACAGGGGGTCTGCAGAGAGGGGTAG |
| | | | | GCGATTGGGTCCGTGGGGGTCATGCCG[CG]GAGT |
| | | | | TTCAAGGACACCCATGGCCCTGAATGGGACTGACC |
| | | | | CTGTTGAGTAATGGGCCATCC (SEQ ID NO: 12) |
| EPB41L3_ | chr18 | 5599586 | − | GACTGGATCATGCATTTGTTTCTCATGAAAGGGTT |
| CpG_438 | | | | AACATCATCCCCTTAGTGCTGTTCT[CG]TGATAGTG |
| | | | | AATGAGCTCTCACAAGTGCTGGTTGTTTGAAAGTA |
| | | | | TGTTGCACCTTCCCCCT (SEQ ID NO: 13) |

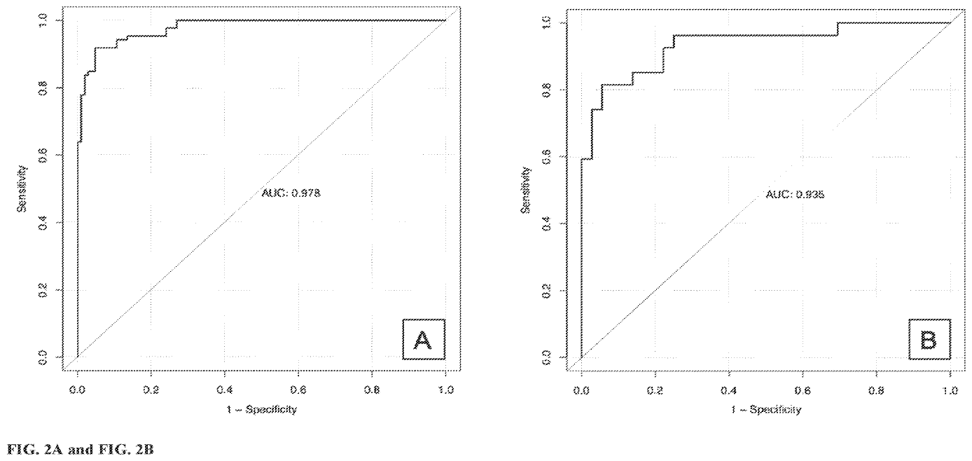

The panel built from the five-step process was used to train the final logistic regression model on all of the data (Table 4). This model was able to predict early OPC compared to controls with an AUC of 0.978 on the training data (FIG. 2a). In the re-sampling validation, an average AUC of 0.935 was achieved indicating adequate internal validity (FIG. 2b).

TABLE 4
Final logistic regression model trained on all samples

| | Estimate | Std. Error | p-value |
|---|---|---|---|
| (Intercept) | −71.822081 | 19.614904 | 0.00025 |
| cg12176286 | 24.3850502 | 10.7673105 | 0.02352926 |
| cg19367172 | 28.1918534 | 13.0475287 | 0.03071818 |
| cg25191818 | 24.4584826 | 47.3726194 | 0.60564491 |
| EPB41L3_CpG_438 | 46.1673225 | 26.6123813 | 0.08277515 |
| HPV16 | 1.98539322 | 0.79846009 | 0.01289965 |
| cg19487745 | 45.6495502 | 17.374909 | 0.00860588 |
| cg20791412 | −12.213209 | 7.39463667 | 0.09860985 |
| cg11125104 | −2.9524888 | 12.7962859 | 0.81752446 |
| cg27131607 | 3.24530032 | 10.8343071 | 0.76452862 |
| cg14500050 | 3.1103516 | 13.4858661 | 0.81759614 |
| cg14705778 | −0.0756728 | 11.5413349 | 0.99476857 |
| cg04025917 | 6.14221486 | 8.70125586 | 0.48025045 |
| cg06459104 | −5.7165068 | 3.54480218 | 0.1068217 |
| cg00619915 | 3.57828823 | 11.3579912 | 0.75272683 |

OPC, a rare event, requires high specificity to minimize false positives. With the 14-marker panel modeled on all samples, a specificity of 99.1% was achieved for prediction of early OPC, with a sensitivity of 70.1%. Allowing specificity to drop to 98.1% resulted in a substantial gain in sensitivity to 78.0%. To account for differences in smoking, it was investigated as a covariate in the model with little change in the outcome (AUC=0.964). We also tested the panel separately in ever vs. never smokers, yielding only slightly better performance in ever smokers (AUC=0.979) compared to never smokers (AUC=0.960).

### c) Discussion

In this study aimed at identifying additional biomarkers to detect early OPC, we were able to improve ROC estimates from 0.78 to an AUC of 0.978 when using training data, and 0.935 when validated with re-sampling methods in the same population. These results demonstrate that addition of several new differentially methylated CpG sites to our biomarker panel significantly improves detection of early OPC in a clinically feasible way by using an easy to collect sample, the oral gargle.

Until recently OPC was considered a rare cancer. However, it is now rapidly increasing in incidence, particularly among men, and is projected to continue to do so for several decades. Therefore, early detection is now a priority at this anatomical site. OPC can present as a neck mass, dysphagia, otalgia, dysphonia, or sore throat, although it is also likely to have no outward physical changes to indicate a cancer diagnosis. Nodal metastases is common at diagnosis due to the rich lymphatics of the oropharynx. However, diagnosis may be delayed due to similarities with other benign conditions afflicting this anatomic region. The American Joint Committee on Cancer (AJCC) staging system was amended with the 8^{th} edition to reflect HPV-positive and HPV-negative OPC as two distinct entities with separate staging parameters because of recent understanding in the differences in molecular features, tumor characteristics, and prognosis. A dependence of HPV-transformed cells on the p16 tumor suppressor, has led to the use of p16 overexpression as measured via immunohistochemistry (IHC) as a surrogate marker to determine if the OPC is associated with HPV. Regardless of HPV attribution, treatment may include radiotherapy, surgery, and chemotherapy. Current standard of care for later stage tumors includes 70 Gy of radiotherapy with concurrent chemotherapy, though while curative in the majority of cases may lead to significant impacts on quality of life, including dysgeusia, xerostomia, dysphagia, soft tissue fibrosis, and rarely neuropathies, osteoradionecrosis or other significant sequelae. Therefore, there is significant interest in de-escalation of therapy with minimally invasive surgical techniques, decreasing radiotherapy dose, or altering/reducing/eliminating systemic therapy. However, as most cases are diagnosed at a stage where multi-modality therapy is necessary, there is a continued need to identify and validate biomarkers to improve early detection of OPC.

A wide array of work has been done to investigate biomarkers for oral cancers utilizing techniques in an “omics” approach-genomics, transcriptomics, proteomics, and metabolomics. The use of “omics” has gained popularity due to its ability to better perform in high dimensional biological settings and to explore the details of the condition in which they are investigating. For example, using metabolomics, five salivary biomarkers were successfully isolated for early diagnosis of OSCC and may represent candidate biomarkers for screening. In this study we investigate changes in the epigenome, to identify differentially methylated CpG sites capable of predicting early oropharyngeal cancer specifically. DNA methylation (DNAm) has been informative in recent studies for diagnosis, as well as prognosis and stratification. DNAm has been used clinically and now in diagnostic kits for specific genes, such as SHOX2 for lung cancer or GSTP1 for prostate cancer. Similar to what we have done in our study, multiple DNAm biomarkers (GSTP1, APC, RASSF1, PTGS2, and MDR1) as a panel were used to achieve sensitivity and specificity near 100% for prostate cancer. However, these studies have relied on a prior knowledge for targeted biomarker identification. New technologies using genome-wide profiles, including the Illumina 850K methylation array, provide an unbiased screen in the exploratory phase of biomarker discovery in which multiple combinations can be evaluated to increase sensitivity and specificity, the approach we have taken in this study.

The epigenetic process of DNAm is a promising biomarker for OPC early detection of lung cancer and colorectal cancer. It has long been known that alternation in DNAm is a hallmark of cancer, and specifically that DNAm of tumor suppressor genes leads to gene repression and uncontrolled carcinogenesis. Recent studies also cite DNAm alterations in pre-cancer. In our prior work towards OPC biomarker development, we applied what was known at the cervix using the EPB41L3 tumor suppressor gene to find that (1) levels of DNAm of EPB41L3 were correlated between OPC tumor specimens and an oral gargle sample; and (2) that DNAm of EPB41L3 measured in the oral gargle can distinguish cases from controls, but better predicted late OPC cases from controls. In this study, we expanded our knowledge to identify additional markers towards creating a biomarker panel for detection of early oropharyngeal cancer.

Within the framework from the Early Detection Research Network (EDRN), our study progresses the work from Phase 1 to Phase 2. Phase 1 is defined as preclinical exploratory studies which aim to find leads for potentially useful biomarkers and prioritize those biomarkers. Phase 2, clinical assay development for clinical disease, is aimed at estimated sensitivity and specificity using an ROC curve to distinguish cases from controls. The results of this study achieve both phases by identifying biomarker leads and identifying a panel to distinguish early OPC from controls. Ultimately, the panel can be employed in dental clinics in a model similar to other cancers detected early (i.e. oral/mouth cancer). A defined at-risk population can be used to identify those in need of screening. An oral gargle can be easily collected at routine dental cleanings and tested with a targeted methylation panel rather than genome-wide array used in this study. Those identified at risk for OPC can be sent for further evaluation.

In our study population, >90% of cases were HPV-positive, which is slightly more than the disease attribution to HPV in the U.S. population of 80%. The biomarker panel disclosed herein can be used in OPC with and without HPV to determine if it can be used to screen for both. At this time, external validation in an independent validation cohort was not possible and so note done.

Specimens from cases were collected at diagnosis and prior to any treatment for OPC. They are, therefore, representative of specimens that can be collected in a screening population. Though other interventions may have taken place including biopsies, change in diet, use of supplements, or other oral health changes. Specimens were also from cases of a primary OPC diagnosis only, making findings particularly relevant to an OPC diagnosis and without confounding from previous cancer diagnosis or treatment. Finally, and perhaps most important to biomarker development, is that this development was done using an oral gargle—a sample that is easy to obtain in multiple settings, making it particularly useful for screening for early OPC.

In conclusion, utilizing data from our prior studies, a fundamental bioinformatics approach, and a simple method to obtain specimens, we have identified a potential biomarker panel to distinguish early OPC that can be implemented in many settings. Unlike other HPV-associated cancers, there is no identifiable precursor lesion prior to cancer development in the oropharynx. Therefore, a reliance on primary prevention via HPV vaccination and early cancer detection of OPC are crucial to improving survival and quality of life. Future work is needed to externally validate this panel and expand it to broader populations. By following the biomarker development framework, the biomarker panel described here can be used to screen at-risk individuals for early OPC, and prevent the intensive systemic therapy required for late OPC diagnoses.

### Oral Gargle Collection

1) If applicable, instruct subject to remove:
   a. Gum from mouth as early in the study visit as possible.
   b. Dentures from mouth prior to specimen collection.
2) Label a 50 ml conical tube with the participants ID (label should be printed out from the tracking system).
3) Fill the 50 ml conical tube with 15 ml of mouthwash (Scope or similar).
4) Ask patient to perform energic washing of oral cavity including the throat by swishing the mouthwash from this container in his mouth vigorously for approximately 15 seconds. Instruct the subject to try to cover all surfaces of his mouth. Next, ask subject to tip their head back and gargle in the throat for another 15 seconds. If patient is not able to gargle in the throat ask him to vigorously swish the mouthwash for 30 seconds.
5) Ask the subject to spit the mouthwash back into the tube.
6) Replace the lid and place specimen in refrigerator until processing (process within 24 hours).

### Standard Operating Procedures

I. SUBJECT: Processing and storage of oral specimen collection
II. OBJECTIVE: To ensure that all oral specimens are processed appropriately
III. RESPONSIBILITY: Clinical Research Coordinator
IV. FREQUENCY: When specimen collected
V. NECESSARY EQUIPMENT:
   i. 15 ml oral rinse specimen in 50 ml conical tube
   ii. Centrifuge (capable of maintaining 4 degrees Celsius)
   iii. Motorized pipet filler/dispenser
   iv. 5 ml serological pipet
   v. 25 ml serological pipet
   vi. 1000 ul pipette
   vii. 41.3 ml PBS (w/o Mg, w/o Ca), refrigerated at 4 degrees Celsius)
   viii. 2.0 ml cryovials
   ix. 2.0 ml Microcentrifuge tubes
   x. 9×9 storage boxes
   xi. Access to Lab Vantage
   xii. −80 degrees Celsius freezer
VI. PROCEDURE:
   Oral Specimen should be Processed within 24 Hours of Collection. If Specimen is not Processed within 1 Hour of Collection, it should be Refrigerated at 4 Degrees Celsius.
i. Centrifuge the oral rinse in the 50 ml conical tube at 2000×g for 15 minutes at 4 degrees Celsius.
ii. Using a Sharpie, write the patient's MRN on three (3) 2.0 ml cryovials, one (1) 2.0 ml microcentrifuge tube, and three (3) 2.0 ml cryovials.
iii. Pipette 1.8 ml supernatant into the three (3) 2.0 ml cryovials.
iv. Discard the remaining supernatant.
v. Re-suspend pellet in 20 ml of cold PBS (4 degrees Celsius)
vi. Mix by inversion 10 times or by using a pipette to assure sample is thoroughly re-suspended.
vii. Repeat centrifugation at 2000×g for 15 minutes at 4 degrees Celsius.
viii. Discard supernatant
ix. Repeat steps v., vi., vii., and viii
x. Re-suspend oral pellet in 1.3 ml of cold PBS (4 degrees Celsius).
xi. Aliquot sample:
   a. 500 ul into a 2.0 ml microcentrifuge tube.
   b. 300 ul into a 2.0 ml cryovial tube.
   c. 250 ul into a 2.0 ml cryovial tube (will be used for microbiome).
   d. 250 ul into a 2.0 ml cryovial tube (will be used for p16).
xii. Enter all aliquoted specimens into Lab Vantage (See Entering Data into Lab Vantage SOP).
xiii. Print labels and place them on the appropriate tubes.
xiv. File all specimens in the appropriate 9×9 storage box and place in the −80 degrees Celsius freezer located in Dr. Giuliano's lab.
xv. After processing the discarded supernatant can be poured down the sink.
VII. DOCUMENTATION:
   i. Lab Vantage ii. Patient's audit sheet

### REFERENCES

Storey J D, Bass A J, Dabney A, Robinson D (2022). qvalue: Q-value estimationforfalse discovery rate control. R package version 2.28.0
Brentnall A R, Vasiljević N, Scibior-Bentkowska D, Cadman L, Austin J, Szarewski A, et al. A DNA methylation classifier of cervical precancer based on human papillomavirus and human genes. International journal of cancer. 2014; 135(6):1425-32.
Chaturvedi A K, Anderson W F, Lortet-Tieulent J, Curado M P, Ferlay J, Franceschi S, et al. Worldwide trends in incidence rates for oral cavity and oropharyngeal cancers. Journal of clinical oncology. 2013; 31(36):4550.
Chaturvedi A K, Engels E A, Pfeiffer R M, Hernandez B Y, Xiao W, Kim E, et al. Human papillomavirus and rising oropharyngeal cancer incidence in the United States. Journal of clinical oncology. 2011; 29(32):4294.
Craig S G, Anderson L A, Schache A G, Moran M, Graham L, Currie K, et al. Recommendations for determining HPV status in patients with oropharyngeal cancers under TNM8 guidelines: a two-tier approach. British Journal of Cancer. 2019; 120(8):827-33.
Delpu Y, Cordelier P, Cho W C, Torrisani J. DNA methylation and cancer diagnosis. International journal of molecular sciences. 2013; 14(7):15029-58.
Dickey B L, Nedjai B, Preece M D, Schell M J, Boulware D, Whiting J, et al. Methylation of HPV16 and EPB41L3 in oral gargles and the detection of early and late oropharyngeal cancer. Cancer Medicine. 2022; 11(20):3735-42.
Dietrich D, Hasinger O, Liebenberg V, Field J K, Kristiansen G, Soltermann A. DNA methylation of the homeobox genes PITX2 and SHOX2 predicts outcome in non-small-cell lung cancer patients. Diagnostic Molecular Pathology. 2012; 21(2):93-104.
Dirks R A, Stunnenberg H G, Marks H. Genome-wide epigenomic profiling for biomarker discovery. Clinical epigenetics. 2016; 8(1):1-17.
Eisbruch A, Harris J, Garden A S, Chao C K, Straube W, Harari P M, et al. Multi-institutional trial of accelerated hypofractionated intensity-modulated radiation therapy for early-stage oropharyngeal cancer (RTOG 00-22). International Journal of Radiation Oncology* Biology* Physics. 2010; 76(5):1333-8.
Faraji F, Eisele D W, Fakhry C. Emerging insights into recurrent and metastatic human papillomavirus-related oropharyngeal squamous cell carcinoma. Laryngoscope investigative otolaryngology. 2017; 2(1):10-8.
Ferris R L, Flamand Y, Weinstein G S, Li S, Quon H, Mehra R, et al. Transoral robotic surgical resection followed by randomization to low- or standard-dose IMRT in resectable p16+ locally advanced oropharynx cancer: a trial of the ECOG-ACRIN Cancer Research Group (E3311). American Society of Clinical Oncology; 2020.
Fortin J-P, Labbe A, Lemire M, Zanke B W, Hudson T J, Fertig E J, et al. Functional normalization of 450 k methylation array data improves replication in large cancer studies. Genome biology. 2014; 15(11):1-17.
Frakes J M, Naghavi A O, Demetriou S K, Strom T J, Russell J S, Kish J A, et al. Determining optimal follow-up in the management of human papillomavirus-positive oropharyngeal cancer. Cancer. 2016; 122(4):634-41.
Fujiwara K, Fujimoto N, Tabata M, Nishii K, Matsuo K, Hotta K, et al. Identification of epigenetic aberrant promoter methylation in serum DNA is useful for early detection of lung cancer. Clinical Cancer Research. 2005; 11(3):1219-25.
Gillison M L, Trotti A M, Harris J, Eisbruch A, Harari P M, Adelstein D J, et al. Radiotherapy plus cetuximab or cisplatin in human papillomavirus-positive oropharyngeal cancer (NRG Oncology RTOG 1016): a randomised, multicentre, non-inferiority trial. The Lancet. 2019; 393(10166):40-50.
Giuliano A R, Nedjai B, Lorincz A T, Schell M J, Rahman S, Banwait R, et al. Methylation of HPV 16 and EPB41L3 in oral gargles: Associations with oropharyngeal cancer detection and tumor characteristics. International journal of cancer. 2020; 146(4):1018-30.
Khalid M B, Ting P, Pai A, Russo J L, Bakst R, Chai R L, et al. Initial presentation of human papillomavirus-related head and neck cancer: A retrospective review. The Laryngoscope. 2019; 129(4):877-82.
Kleter B, Van Doom L-J, Schrauwen L, Molijn A, Sastrowijoto S, ter Schegget J, et al. Development and clinical evaluation of a highly sensitive PCR-reverse hybridization line probe assay for detection and identification of anogenital human papillomavirus. Journal of clinical microbiology. 1999; 37(8):2508-17.
Kreimer A R, Villa A, Nyitray A G, Abrahamsen M, Papenfuss M, Smith D, et al. The epidemiology of oral HPV infection among a multinational sample of healthy men. Cancer epidemiology, biomarkers & prevention. 2011; 20(1):172-82.
Lechner M, Liu J, Masterson L, Fenton T R. HPV-associated oropharyngeal cancer: Epidemiology, molecular biology and clinical management. Nature reviews Clinical oncology. 2022; 19(5):306-27.
Marur S, D'Souza G, Westra W H, Forastiere A A. HPV-associated head and neck cancer: a virus-related cancer epidemic. The lancet oncology. 2010; 11(8):781-9.
McIlwain W R, Sood A J, Nguyen S A, Day T A. Initial symptoms in patients with HPV-positive and HPV-negative oropharyngeal cancer. JAMA Otolaryngology—Head & Neck Surgery. 2014; 140(5):441-7.
Müller H M, Oberwalder M, Fiegl H, Morandell M, Goebel G, Zitt M, et al. Methylation changes in faecal DNA: a marker for colorectal cancer screening? The Lancet. 2004; 363(9417):1283-5.
Pepe M S, Etzioni R, Feng Z, Potter J D, Thompson M L, Thornquist M, et al. Phases of biomarker development for early detection of cancer. Journal of the National Cancer Institute. 2001; 93(14):1054-61.
Pepe M S, Janes H, Li C I, Bossuyt P M, Feng Z, Hilden J. Early-phase studies of biomarkers: what target sensitivity and specificity values might confer clinical utility? Clinical chemistry. 2016; 62(5):737-42.
Rai V, Mukherjee R, Ghosh A K, Routray A, Chakraborty C. “Omics” in oral cancer: New approaches for biomarker discovery. Archives of Oral Biology. 2018; 87:15-34.
Robin X, Turck N, Hainard A, Tiberti N, Lisacek F, Sanchez J-C, et al. pROC: an open-source package for R and S+ to analyze and compare ROC curves. BMC bioinformatics. 2011; 12(1):1-8.
Storey J, Bass A, Dabney A, Robinson D. 2019. Qvalue: q-value estimation for false discovery rate control. R package version 2.16. 0. 2022.
Storey J D. A direct approach to false discovery rates. Journal of the Royal Statistical Society Series B: Statistical Methodology. 2002; 64(3):479-98.
Wang Q, Gao P, Wang X, Duan Y. The early diagnosis and monitoring of squamous cell carcinoma via saliva metabolomics. Scientific Reports. 2014; 4(1):6802.
Wu T, Giovannucci E, Welge J, Mallick P, Tang W, Ho S. Measurement of GSTP1 promoter methylation in body fluids may complement PSA screening: a meta-analysis. British journal of cancer. 2011; 105(1):65-73.
Yegnasubramanian S, Kowalski J, Gonzalgo M L, Zahurak M, Piantadosi S, Walsh P C, et al. Hypermethylation of CpG islands in primary and metastatic human prostate cancer. Cancer research. 2004; 64(6):1975-86.
Yom S S, Torres-Saavedra P, Caudell J J, Waldron J N, Gillison M L, Xia P, et al. Reduced-dose radiation therapy for HPV-associated oropharyngeal carcinoma (NRG Oncology HN002). Journal of Clinical Oncology. 2021; 39(9):956.

1. An assay for the detection of oropharyngeal cancer in a subject comprising probes to detect methylation at CpG sites in one or more genes in a tissue sample;
wherein the genes comprise PAPD5, ST8SIA5, SNORD115-20, NCRNA00164; MIR663B, DDX42, RASA3, HMGB2, ARMC6, EPB41L3; and
wherein a change in the amount of methylation of 3 or more CpG sites relative to a normal control indicates the presence of oropharyngeal cancer.

2. The assay of claim 1, wherein the detection of CpG sites is accomplished using the CpG probes cg25191818, cg19367172, cg12176286, cg19487745, cg20791412, cg11125104, cg27131607, cg14500050, cg14705778, cg04025917, cg06459104, and cg00619915.

3. The assay of claim 1, further comprising a probe for the detection of EPB41L3 CpG site 438.

4. The assay of claim 1, further comprising an HPV SPF₁₀ PCR-DEIA-LiPA₂₅ line probe for the detection of a human papilloma virus 16 (HPV16) infection or a human papilloma virus 18 (HPV18) infection.

5. A method of detecting oropharyngeal cancer in a subject, comprising:
(i) obtaining a tissue sample from the subject; and
(ii) applying the tissue sample to the assay of claim 1.

6. A method of detecting oropharyngeal cancer in a subject, comprising:
(i) obtaining a tissue sample from the subject; and
(ii) measuring the methylation at CpG sites of one or more genes comprising PAPD5, ST8SIA5, SNORD115-20, NCRNA00164; MIR663B, DDX42, RASA3, HMGB2, ARMC6, and/or EPB41L3;
wherein a change in the amount of methylation of 3 or more CpG sites relative to a normal control indicates the presence of oropharyngeal cancer.

7. The method of claim 6, wherein the detection of CpG sites is accomplished using the CpG probes cg25191818, cg19367172, cg12176286, cg19487745, cg20791412, cg11125104, cg27131607, cg14500050, cg14705778, cg04025917, cg06459104, and cg00619915.

8. The method of claim 6, further comprising a probe for the detection of EPB41L3 CpG site 438.

9. The method of claim 6 wherein the tissue sample is obtained via oral wash or gargle.

10. The method of claim 6 further comprising assaying the sample for the presence of a human papilloma virus 16 (HPV16) infection or a human papilloma virus 18 (HPV18) infection.

11. The method of claim 10 wherein HPV16 and/or HPV18 infection is detected using an HPV SPF₁₀ PCR-DEIA-LiPA₂₅ line probe.

12. A method of treating oropharyngeal cancer in a subject comprising:
(i) obtaining a tissue sample from the subject;
(ii) applying the tissue sample to the assay of claim 1; and
(iii) treating the oropharyngeal cancer when detected.

13. A method of treating oropharyngeal cancer in a subject comprising:
i) obtaining a tissue sample from the subject;
ii) measuring the methylation at CpG sites of one or more genes comprising PAPD5, ST8SIA5, SNORD115-20, NCRNA00164; MIR663B, DDX42, RASA3, HMGB2, ARMC6, and/or EPB41L3;
wherein a change in the amount of methylation of 3 or more CpG sites relative to a normal control indicates the presence of oropharyngeal cancer; and
iii) treating the oropharyngeal cancer when detected.

14. The method of claim 13, wherein the cancer is treated with the administration of an anti-cancer agent and/or radiation.

15. The method of claim 13, wherein the detection of CpG sites is accomplished using the CpG probes cg25191818, cg19367172, cg12176286, cg19487745, cg20791412, cg11125104, cg27131607, cg14500050, cg14705778, cg04025917, cg06459104, and cg00619915.

16. The method of claim 13, further comprising a probe for the detection of EPB41L3 CpG site 438.

17. The method of claim 13, wherein the tissue sample is obtained via oral wash or gargle.

18. The method of claim 13, further comprising assaying the sample for the presence of a human papilloma virus 16 (HPV16) infection or a human papilloma virus 18 (HPV18) infection.

19. The method of claim 18, wherein HPV16 and/or HPV18 infection is detected using an HPV SPF₁₀ PCR-DEIA-LiPA₂₅ line probe.


SEQUENCE LISTING (13 sequences)

SEQ ID NO: 1 - DNA, 121 bp, synthetic construct
  misc_feature 1..121: note = Description of sequence: DNA probe
  source 1..121: mol_type = other DNA
cttgatctcttgacctcgtgatccgcctgccttggccttccaaagtgctgggattacaggstgagccaccgcggccggccatttttgttgttgttgttgttgttgttgttgttgttgttga

SEQ ID NO: 2 - DNA, 121 bp, synthetic construct
  misc_feature 1..121: note = Description of sequence: DNA probe
  source 1..121: mol_type = other DNA
tttgccccgcggcttgttggggtctcctttacccatgttgacagatccgcgtccacctgasgggccgaggggggagaggggaagccggagggtcggcgcggagcccgcagctgccagggcg

SEQ ID NO: 3 - DNA, 121 bp, synthetic construct
  misc_feature 1..121: note = Description of sequence: DNA probe
  source 1..121: mol_type = other DNA
aggcgaggtggggcttttgggaaaattacataatccccgcccctgggccggaactgccctscctatcccaatcctcctacctaggattccgtcaagttgacgtcacacggaaccggtgctc

SEQ ID NO: 4 - DNA, 121 bp, synthetic construct
  misc_feature 1..121: note = Description of sequence: DNA probe
  source 1..121: mol_type = other DNA
gctgccgaggcctgtcccgcgcggagtggggagtgggggtgccgcagggtcgggctggcgsaccctcgggtacgtggtttggggggaaaaaaaaaatgaaagaattttacagaagccgcct

SEQ ID NO: 5 - DNA, 121 bp, synthetic construct
  misc_feature 1..121: note = Description of sequence: DNA probe
  source 1..121: mol_type = other DNA
ggcggggcacacgtagccgtgcagggtctcgcgcagcccgcgtcaggaccccggggcacasctcgggcagggggcgtgtccccacgccaccagaaaaagtgttataggctgtttttttttt

SEQ ID NO: 6 - DNA, 121 bp, synthetic construct
  misc_feature 1..121: note = Description of sequence: DNA probe
  source 1..121: mol_type = other DNA
gcacacctctcagatcgctagagaaggctttctcaccgagggtgggtcacactccccacasccagtcgcccctcgtcgggcccgcagaggcgctcagggacgcctggggaagggagggggc

SEQ ID NO: 7 - DNA, 121 bp, synthetic construct
  misc_feature 1..121: note = Description of sequence: DNA probe
  source 1..121: mol_type = other DNA
ttccacctcacagcagttgtatccatttcttcattgtgtctcttcctgttacaatccctgscgcacacacatagtcctaccttcaattatttattcattatccaatatttattgagaacat

SEQ ID NO: 8 - DNA, 121 bp, synthetic construct
  misc_feature 1..121: note = Description of sequence: DNA probe
  source 1..121: mol_type = other DNA
tcctcgcggggaaatcccggggaagtgctgagtctggagtatcgactcccttcgtcctcgsgggaaatcacggggaagtgctgagtctggagtatttattcccttcgtcctcgcggggaaa

SEQ ID NO: 9 - DNA, 121 bp, synthetic construct
  misc_feature 1..121: note = Description of sequence: DNA probe
  source 1..121: mol_type = other DNA
tttttttttttgagacagagtctcactctgttgcttgactggagtgcattggcatgatctsgctcactgcaacctctgcttcccgggttcaagcgattctcctgcctcagcctcctgagta

SEQ ID NO: 10 - DNA, 121 bp, synthetic construct
  misc_feature 1..121: note = Description of sequence: DNA probe
  source 1..121: mol_type = other DNA
gacaggggtaaggaggaagctgctgaaccctgctaagaagatgggaggcctggtctaattsagaaccagacacggcaggccctgtcttctgggctcagcccagtgagaagctggagcagca

SEQ ID NO: 11 - DNA, 121 bp, synthetic construct
  misc_feature 1..121: note = Description of sequence: DNA probe
  source 1..121: mol_type = other DNA
tcatccgacaccagccgcctccaccatgccgccgaagttccaccccaatgagatcaaagtstatacttcacgtgcactggagttgaagtcggtgccacttctgctctggcccccaagattg

SEQ ID NO: 12 - DNA, 121 bp, synthetic construct
  misc_feature 1..121: note = Description of sequence: DNA probe
  source 1..121: mol_type = other DNA
ggggcactacagggggtctgcagagaggggtaggcgattgggtccgtgggggtcatgccgsgagtttcaaggacacccatggccctgaatgggactgaccctgttgagtaatgggccatcc

SEQ ID NO: 13 - DNA, 121 bp, synthetic construct
  misc_feature 1..121: note = Description of sequence: DNA probe
  source 1..121: mol_type = other DNA
gactggatcatgcatttgtttctcatgaaagggttaacatcatccccttagtgctgttctstgatagtgaatgagctctcacaagtgctggttgtttgaaagtatgttgcaccttccccct